(19) United States
(12) Patent Application Publication
(10) Pub. No.: US 2025/0258088 A1
(43) Pub. Date: Aug. 14, 2025

(54) CONCENTRATION MEASUREMENT DEVICE

(71) Applicant: Sony Group Corporation, Tokyo (JP)

(72) Inventor: Hideya Chubachi, Kanagawa (JP)

(73) Assignee: Sony Group Corporation, Tokyo (JP)

(21) Appl. No.: 18/848,212

(22) PCT Filed: Oct. 11, 2022
(86) PCT No.: PCT/JP2022/037810

§ 371 Date: Apr. 29, 2025

(30) Foreign Application Priority Data
Mar. 31, 2022 (JP) 2022-058419

(51) Int. Cl. G01N 21/31; G01N 33/49; G05D 11/13

(52) U.S. Cl. CPC G01N 21/31; G01N 33/49; G05D 11/138; G01N 2021/3129; G01N 2201/12746

(57) ABSTRACT

To improve measurement accuracy.

A concentration measurement device includes: an absorbance measurement unit configured to measure absorbance in a plurality of different wavelength regions for a mixed liquid in which different liquids are mixed; and a control unit configured to correct a concentration of at least one liquid on the basis of absorbance in the plurality of different wavelength regions. The absorbance measurement unit measures absorbance in two wavelength regions. The absorbance measurement unit measures absorbance in a spectrum. Particles contained in one of the different liquids are different from particles contained in another of the different liquids. Each of the different liquids is a suspension or a solution. The mixed liquid includes a white blood cell suspension and a red blood cell suspension. The control unit calculates a mixing ratio of a liquid mixed in the mixed liquid, from absorbance in the plurality of different wavelength regions. The control unit corrects the absorbance on the basis of a mixing ratio of a liquid mixed in the mixed liquid.

### TECHNICAL FIELD

The present technology relates to a concentration measurement device.

### BACKGROUND ART

Conventionally, there is known a concentration measurement method for a mixed solution component, in which a mixed solution containing a plurality of biological components as measurement target substances is irradiated with infrared light, absorbance is detected by Fourier transform infrared spectroscopy, and concentrations of the plurality of measurement target substances contained in the mixed solution are determined (see, for example, Patent Document 1).

### CITATION LIST

### Patent Document

Patent Document 1: Japanese Patent Application Laid-Open No. 2005-147896

### SUMMARY OF THE INVENTION

### Problems to be Solved by the Invention

However, in the concentration measurement method for a mixed solution component described in Patent Document 1, in a case where a concentration measurement target is a cell suspension, the cell suspension may contain impurities. Furthermore, since a rate of the contained impurities varies for each measurement target and absorbance varies depending on an amount of the contained impurities, a cell concentration of the cell suspension may not be correctly calculated with a calibration curve prepared in advance.

A main object of the present technology is to provide a concentration measurement device capable of improving measurement accuracy.

### Solutions to Problems

The present technology provides a concentration measurement device including:
an absorbance measurement unit configured to measure absorbance in a plurality of different wavelength regions for a mixed liquid in which different liquids are mixed; and
a control unit configured to correct a concentration of at least one liquid on the basis of absorbance in the plurality of different wavelength regions.

The absorbance measurement unit can measure absorbance in two wavelength regions.

The absorbance measurement unit can measure absorbance in a spectrum.

Particles contained in one of the different liquids can be different from particles contained in another of the different liquids.

Each of the different liquids can be a suspension or a solution.

The mixed liquid can contain a white blood cell suspension and a red blood cell suspension.

The control unit can calculate a mixing ratio of a liquid mixed in the mixed liquid, from absorbance in the plurality of different wavelength regions.

The control unit can correct the absorbance on the basis of a mixing ratio of a liquid mixed in the mixed liquid.

The concentration measurement device can further include a calibration curve creation unit configured to create a calibration curve by using a calibration-curve-creation mixed liquid in which a component is same as a component of the mixed liquid and a concentration of each mixed liquid is known.

The calibration curve creation unit can create a calibration curve for obtaining a concentration of at least one mixed liquid, on the basis of absorbance in a specific wavelength region by using the calibration-curve-creation mixed liquid in which a concentration of the at least one liquid is known.

The calibration curve creation unit can create a calibration curve for obtaining a mixing ratio of a liquid mixed in the mixed liquid, on the basis of a difference in absorbance in the plurality of different wavelength regions.

The calibration curve creation unit can create a calibration curve for obtaining absorbance on the basis of a mixing ratio of a liquid mixed in the mixed liquid.

The concentration measurement device according to the present technology can further include:
a light source configured to emit light;
a light-transmissive container inside of which a fluid containing the mixed liquid flows; and
a detection unit configured to detect light having passed through the container.

The light source can include: a first light source configured to emit light having a first wavelength; and
a second light source configured to emit light having a second wavelength different from the first wavelength.

The present technology provides a concentration measurement method including:
an absorbance measuring step of measuring absorbance in a plurality of different wavelength regions for a mixed liquid in which different liquids are mixed; and
a correction step of correcting a concentration of at least one liquid on the basis of absorbance in the plurality of different wavelength regions.

The concentration measurement method can further include a calibration curve creating step of creating a calibration curve by using a calibration-curve-creation mixed liquid in which a component is same as a component of the mixed liquid and a concentration of each mixed liquid is known.

### BRIEF DESCRIPTION OF DRAWINGS

FIG. 1 is a diagram illustrating a configuration of a concentration adjustment device including a concentration measurement device according to a first embodiment of the present technology.

FIG. 2 is a diagram illustrating a configuration of an absorbance measurement unit in the concentration measurement device according to the first embodiment of the present technology.

FIG. 3 is a perspective view of a tube holder as viewed from one side.

FIG. 4 is a perspective view of the tube holder as viewed from a direction in which a surface opposite to a surface of the tube holder visible in FIG. 3 is visible.

FIG. 5 is a diagram illustrating another configuration of the absorbance measurement unit in the concentration measurement device according to the first embodiment of the present technology.

FIG. 6 is a diagram illustrating a calculation method for a concentration of a solution containing a substance having Concentration C.

FIG. 7 is a graph illustrating a relationship between Absorbance A and Concentration C.

FIG. 8 is a graph illustrating an absorbance spectrum of Solution 1.

FIG. 9 is a graph illustrating an absorbance spectrum of Solution 2.

FIG. 10 is a graph illustrating an absorbance spectrum of a mixed liquid of Solution 1 and Solution 2.

FIG. 11 is a graph illustrating a relationship between an absorbance difference (A12λ1−A12λ2) at two wavelengths and the mixing ratio (E/D).

FIG. 12 is a graph illustrating a relationship between a mixing ratio (E/D) and absorbance.

FIG. 13 is a diagram illustrating a configuration of a specimen analyzer.

FIG. 14 is a hardware configuration diagram illustrating an example of a computer that implements a function of a control unit in the concentration measurement device according to the first embodiment of the present technology.

FIG. 15 is a graph illustrating an absorbance spectrum at a predetermined RBC/WBC ratio.

FIG. 16 is a graph illustrating Calibration curve (1).

FIG. 17 is a photograph showing a sample used for creating Calibration curve (2).

FIG. 18 is a graph illustrating an absorbance spectrum at a predetermined RBC/WBC ratio.

FIG. 19 is a graph illustrating Calibration curve (2).

FIG. 20 is a graph illustrating Calibration curve (3).

FIG. 21 is a graph illustrating an absorbance spectrum of a sample to be measured.

FIG. 22 is a photograph showing a sample used for creating Calibration curve (1).

FIG. 23 is a graph illustrating Calibration curve (1).

FIG. 24 is a photograph showing a sample used for creating Calibration curve (2).

FIG. 25 is a graph illustrating an absorbance spectrum of a sample.

FIG. 26 is a graph illustrating Calibration curve (2) on an RBC basis.

FIG. 27 is a graph illustrating Calibration curve (2) on an HGB basis.

FIG. 28 is a graph illustrating Calibration curve (3) on an RBC basis.

FIG. 29 is a graph illustrating Calibration curve (3) on an HGB basis.

FIG. 30 is a photograph showing a sample used in Verification experiment 1.

FIG. 31 is a photograph showing a sample used in Verification experiment 2.

FIG. 32 is a graph illustrating an absorbance spectrum of a sample used in Verification experiment 1.

FIG. 33 is a graph illustrating an absorbance spectrum of a sample used in Verification experiment 2.

### MODE FOR CARRYING OUT THE INVENTION

Hereinafter, preferred embodiments for carrying out the present technology will be described with reference to the drawings. Note that embodiments described below illustrate representative embodiments of the present technology, and the scope of the present technology is not limited only to these embodiments. Moreover, in the description of the drawings, the same portions are denoted by the same reference numerals.

The present technology will be described in the following order.
1. Description of present technology
2. First embodiment (example of concentration measurement device)
(1) Configuration of concentration adjustment device
(2) Configuration of concentration measurement device
(3) Description of concentration calculation method
(4) Configuration of specimen analyzer
3. Second embodiment (example of concentration measurement method)
(1) Description of concentration measurement method according to present embodiment
(2) Description of each step
4. Examples

### 1. Description of Present Technology

Conventionally, absorbance of a solution is measured, and a solution concentration is calculated according to the Lambert-Beer law. However, a solution contained in a sample to be measured is not necessarily one type, and the sample to be measured may be a mixed liquid of two types of solutions. In this case, even if the concentration of one solution is to be calculated according to the Lambert-Beer law, accurate absorbance cannot be obtained due to an influence of another solution.

The present inventor has found that, in a case of measuring absorbance of a mixed liquid containing mixed different solutions and having characteristics in an absorbance spectrum, a concentration of one solution can be accurately measured by measuring absorbance in a plurality of different wavelengths and correcting the absorbance to absorbance of a case of only one solution.

That is, the concentration measurement device according to the present technology includes: an absorbance measurement unit configured to measure absorbance in a plurality of different wavelength regions for a mixed liquid in which different liquids are mixed; and a control unit configured to correct a concentration of at least one liquid on the basis of absorbance in the plurality of different wavelength regions. Furthermore, the concentration measurement device according to the present technology further includes: a calibration curve creation unit configured to create a calibration curve by using a calibration-curve-creation mixed liquid in which a component is same as a component of the mixed liquid and a concentration of each mixed liquid is known.

Furthermore, the concentration measurement method according to the present technology includes: an absorbance measuring step of measuring absorbance in a plurality of different wavelength regions for a mixed liquid in which different liquids are mixed; and a correction step of correcting a concentration of at least one liquid on the basis of absorbance in the plurality of different wavelength regions.

Moreover, the concentration measurement method according to the present technology further includes: a calibration curve creation step of creating a calibration curve by using a calibration-curve-creation mixed liquid in which a component is same as a component of the mixed liquid and a concentration of each mixed liquid is known.

### 2. First Embodiment (Example of Concentration Measurement Device)

### (1) Configuration of Concentration Adjustment Device

A concentration measurement device according to the present embodiment may be provided in a concentration adjustment device that inputs a mixed liquid to a specimen analyzer. The concentration measurement device may measure a concentration of a mixed liquid in which different liquids are mixed, adjust the concentration of the mixed liquid by using the concentration adjustment device on the basis of the concentration, and input the mixed liquid into the specimen analyzer.

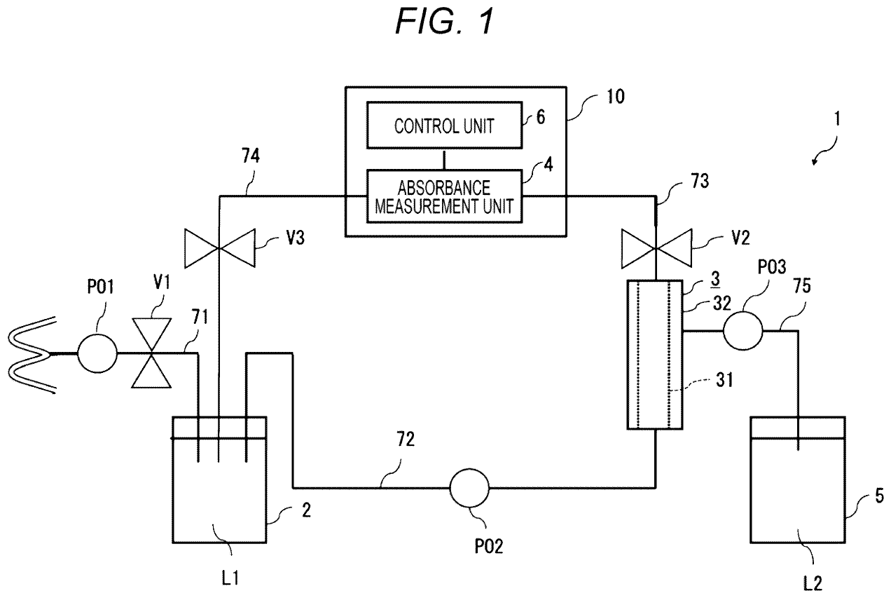

FIG. 1 is a diagram illustrating a configuration of a concentration adjustment device 1 including a concentration measurement device 10 according to the present embodiment.

The concentration adjustment device 1 is a device that adjusts a concentration of a mixed liquid in which different liquids are mixed. Specifically, the concentration adjustment device 1 adjusts a concentration of a liquid to an appropriate concentration that should be input to a specimen analyzer 6100 (see FIG. 13). Note that the concentration adjustment device 1 and the specimen analyzer 6100 may be directly connected by a tube, and the mixed liquid whose concentration has been adjusted by the concentration adjustment device 1 may be input into the specimen analyzer 6100 via the tube. Alternatively, the mixed liquid whose concentration has been adjusted by the concentration adjustment device 1 may be taken out from the concentration adjustment device 1 and input into the specimen analyzer 6100. In the first embodiment, the specimen analyzer 6100 is, for example, a device used for cell therapy, and a detailed configuration thereof will be described in “Configuration of specimen analyzer” described later. Then, in the first embodiment, the mixed liquid is a mixture of different liquids, may be a suspension or a solution, and can contain particles. Furthermore, the mixed liquid may be a cell suspension stained with an antibody dye (labeled with a labeling substance). In a case where the mixed liquid is a cell suspension, the concentration adjustment device 1 aseptically adjusts a cell concentration in the cell suspension to an appropriate cell concentration that should be input to the specimen analyzer 6100 described later, without directly touching the cell suspension. As illustrated in FIG. 1, the concentration adjustment device 1 includes a liquid container 2, a hollow fiber module 3, the concentration measurement device 10, a waste liquid container 5, pipes 71 to 75, pumps PO1 to PO3, and valves V1 to V3. Furthermore, the concentration measurement device 10 includes an absorbance measurement unit 4 and a control unit 6.

As illustrated in FIG. 1, the liquid container 2 is a container that stores a mixed liquid L1 in which different liquids are mixed.

The pipe 71 is connected to the liquid container 2. Then, under the control of the control unit 6 provided in the concentration measurement device 10, when the valve V1 disposed on the pipe 71 is opened and the pump PO1 is driven, the mixed liquid L1 is supplied into the liquid container 2 via the pipe 71.

Furthermore, the pipes 72 and 74 are connected to the liquid container 2. Moreover, the liquid container 2 is disposed on an annular flow channel including the pipe 72, the hollow fiber module 3, the pipe 73, the absorbance measurement unit 4, the pipe 74, the liquid container 2, and the pipe 72. Then, under the control of the control unit 6, when the valve V2 disposed on the pipe 73 and the valve V3 disposed on the pipe 74 are opened, and the pump PO2 disposed on the pipe 72 is driven, the mixed liquid L1 in the liquid container 2 flows through the annular flow channel.

As illustrated in FIG. 1, the hollow fiber module 3 includes a hollow fiber membrane 31 and an outer cylinder 32 accommodating the hollow fiber membrane 31. Note that, although only one hollow fiber membrane 31 is illustrated in the outer cylinder 32 in FIG. 1, in practice, multiple hollow fiber membranes 31 are accommodated in the outer cylinder 32.

The hollow fiber membrane 31 is a membrane formed in a straw shape with a hollow inside, and has a large number of pores smaller than particles contained in the mixed liquid L1 on a surface. For example, in a case where the mixed liquid is a cell suspension, the hole is a hole that allows unbound antibody dye and the like to pass therethrough and does not allow cells to pass therethrough.

The pipe 75 is connected to the hollow fiber module 3. Then, when the pump PO3 disposed on the pipe 75 is driven under the control of the control unit 6, and accordingly the mixed liquid L1 flows through the hollow fiber membrane 31 following the annular flow channel described above, an unbound antibody dye and the like in the mixed liquid L1 are discharged to the outside of the hollow fiber membrane 31 while cells in the mixed liquid L1 remain in the hollow fiber membrane 31. The unbound antibody dye and the like discharged to the outside of the hollow fiber membrane 31 are discharged into the waste liquid container 5 through the pipe 75.

The concentration measurement device 10 is a device for measuring a cell concentration in the mixed liquid L1 flowing through the annular flow channel described above.

Note that a detailed configuration of the concentration measurement device 10 will be described in “Configuration of concentration measurement device” described later.

As illustrated in FIG. 1, the waste liquid container 5 is a container that stores a waste liquid L2 such as unbound antibody dye discharged to the outside of the hollow fiber membrane 31.

### (2) Configuration of Concentration Measurement Device

Next, a configuration of the concentration measurement device 10 will be described. The concentration measurement device 10 includes the absorbance measurement unit 4 and the control unit 6. Furthermore, the concentration measurement device 10 may further include a calibration curve creation unit 7. Hereinafter, the absorbance measurement unit 4, the control unit 6, and the calibration curve creation unit 7 will be described.

### [Configuration of Absorbance Measurement Unit]

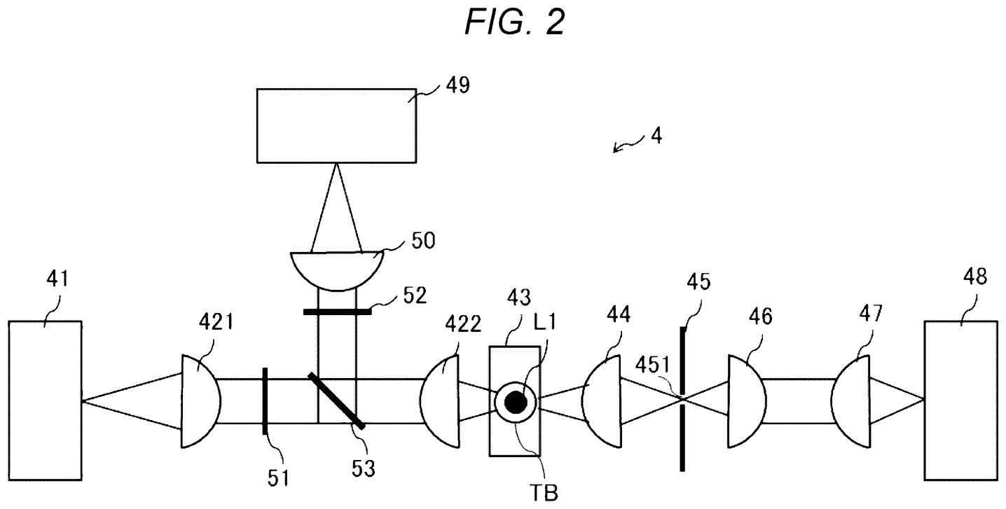

The absorbance measurement unit 4 measures absorbance in a plurality of different wavelength regions for a mixed liquid in which different liquids are mixed. A configuration example of such the absorbance measurement unit 4 is illustrated in FIG. 2. The absorbance measurement unit 4 illustrated in FIG. 2 includes a first light source 41, a first lens 421, a second lens 422, a third lens 44, a light shielding plate 45, a fourth lens 46, and a fifth lens 47, a tube TB, a third lens 44, a fourth lens 46, and a fifth lens 47, a detection unit 48, a second light source 49, a sixth lens 50, a first shutter 51, a second shutter 52, and a dichroic mirror 53.

The tube TB is formed in a cylindrical shape and has optical transparency. As a material of the tube TB, a resin material such as polyvinyl chloride (PVC) can be exemplified. Then, the tube TB constitutes a part of the annular flow channel described above. That is, the mixed liquid L1 flows through the tube TB.

The first light source 41 emits light toward the mixed liquid L1 in the tube TB. In the first embodiment, the first light source 41 emits light in a wavelength region of a wavelength λ1.

As illustrated in FIG. 2, the first lens 421 is disposed on an optical path latter-stage side with respect to the first light source 41, and collimates the light emitted from the first light source 41.

As illustrated in FIG. 2, the first shutter 51 is disposed on an optical path latter-stage side with respect to the first lens 421. The first shutter 51 is a plate body, and is made containing a light shielding material that shields light. Furthermore, the first shutter 51 is disposed in an orientation in which each plate surface is substantially orthogonal to an optical axis of the light emitted from the first light source 41. When the mixed liquid L1 in the tube TB is irradiated with the light from the first light source 41, the first shutter 51 allows parallel light from the first light source 41 to pass therethrough.

As illustrated in FIG. 2, the dichroic mirror 53 is disposed on an optical path between the first lens 421 and the second lens 422. The dichroic mirror 53 is an optical element of a wavelength-selective reflection type, and has a light reflectance different according to a wavelength of light. Each plate surface of the dichroic mirror 53 is disposed at a predetermined angle with respect to the optical axis of the light emitted from the first light source 41. When the mixed liquid L1 in the tube TB is irradiated with the light from the first light source 41, the dichroic mirror 53 allows parallel light from the first light source 41 to pass therethrough.

As illustrated in FIG. 2, the second lens 422 is disposed on an optical path latter-stage side with respect to the first lens 421, and condenses parallel light having passed through the first lens 421 at a position on an optical path former-stage side with respect to the tube TB.

The second light source 49 emits light toward the mixed liquid L1 in the tube TB. In the first embodiment, the second light source 49 emits light of a wavelength region of a wavelength λ2 different from the light emitted from the first light source 41.

As illustrated in FIG. 2, the sixth lens 50 is disposed on an optical path latter-stage side with respect to the second light source 49, and collimates the light emitted from the second light source 49.

As illustrated in FIG. 2, the second shutter 52 is disposed on an optical path latter-stage side with respect to the sixth lens 50. The second shutter 52 is a plate body, and is made containing a light shielding material that shields light. Furthermore, the second shutter 52 is disposed in an orientation in which each plate surface is substantially orthogonal to an optical axis of the light emitted from the second light source 49. When the liquid L1 in the tube TB is irradiated with the light from the second light source 49, the first shutter 51 blocks parallel light from the first light source 41, and the second shutter 52 allows parallel light from the second light source 49 to pass therethrough. When the liquid L1 in the tube TB is irradiated with the light from the first light source 41, the second shutter 52 blocks parallel light from the second light source 49.

When the liquid L1 in the tube TB is irradiated with the light from the second light source 49, the dichroic mirror 53 reflects parallel light from the second light source 49 to pass through the second lens 422.

As illustrated in FIG. 2, the second lens 422 is disposed on an optical path latter-stage side with respect to the dichroic mirror 53, and condenses parallel light having passed through the dichroic mirror 53 at a position on an optical path former-stage side with respect to the tube TB.

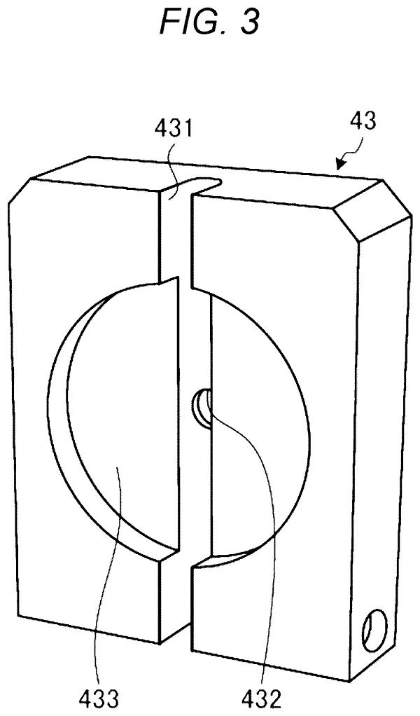

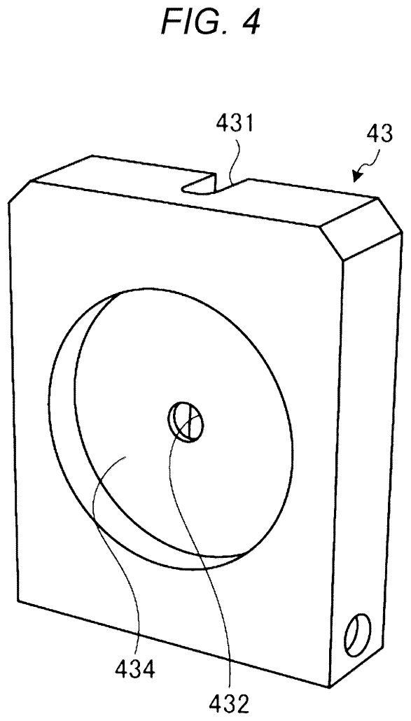

FIGS. 3 and 4 are views illustrating a tube holder 43. Specifically, FIG. 3 is a perspective view of the tube holder 43 as viewed from one side, and FIG. 4 is a perspective view of the tube holder 43 as viewed from a direction in which a surface opposite to a surface of the tube holder 43 visible in FIG. 3 is visible. The surface of the tube holder 43 visible in FIG. 3 may be on the optical path former-stage side or on the optical path latter-stage side. Similarly, the surface of the tube holder 43 visible in FIG. 4 may be on the optical path former-stage side or the optical path latter-stage side.

As illustrated in FIG. 2, the tube holder 43 is disposed on an optical path latter-stage side with respect to the first lens 421 and the second lens 422. As illustrated in FIG. 3 or 4, the tube holder 43 is a plate body having a substantially rectangular shape in plan view, and is made containing a light shielding material that shields light. Then, the tube holder 43 holds the tube TB. Each plate surface of the tube holder 43 is disposed in an orientation in which each plate surface is substantially orthogonal to the optical axis of the light emitted from the first light source 41.

In the tube holder 43, a tube groove 431 linearly extending in a vertical direction in FIG. 3 or 4 is formed on a plate surface on the optical path former-stage side.

Furthermore, the tube holder 43 is formed with a through hole 432 that is located substantially at a center of the plate surface, penetrates each plate surface, and communicates with the tube groove 431.

Moreover, in the tube holder 43, circular recesses 433 and 434 centered on the through hole 432 are formed on each plate surface.

Then, the tube TB is held by the tube holder 43 in a state of being inserted into the tube groove 431. Furthermore, a part of light having passed through the tube TB passes through the through hole 432.

Here, the tube TB collimates light incident on a side surface in a state where the mixed liquid L1 flows inside. Specifically, in the first embodiment, the tube TB functions as an optical element (cylindrical lens) that collimates light transmitted through the tube TB out of light having passed through the first lens 421 and the second lens 422, in a plane orthogonal to a longitudinal direction of the tube TB. That is, a focal position of the tube TB (cylindrical lens) is set at a light condensing position of the second lens 422 (focal position of the second lens 422).

As illustrated in FIG. 2, the light shielding plate 45 is disposed on an optical path latter-stage side with respect to the tube holder 43. The light shielding plate 45 is a plate body, and is made containing a light shielding material that shields light. Furthermore, the light shielding plate 45 is disposed in an orientation in which each plate surface is substantially orthogonal to the optical axis of the light emitted from the first light source 41.

As illustrated in FIG. 2, an opening 451 penetrating each plate surface is formed at a substantially central position of the light shielding plate 45.

As illustrated in FIG. 2, the third lens 44 is provided between the tube TB and the light shielding plate 45. That is, the third lens 44 condenses parallel light having passed through the tube TB on the opening 451.

As illustrated in FIG. 2, the fourth lens 46 is disposed on an optical path latter-stage side with respect to the light shielding plate 45. Then, the fourth lens 46 collimates light condensed by the third lens 44 and having passed through the opening 451.

As illustrated in FIG. 2, the fifth lens 47 is disposed on an optical path latter-stage side with respect to the fourth lens 46. Then, the fifth lens 47 condenses light collimated by the fifth lens 47 on a detection surface of the detection unit 48.

The detection unit 48 detects light having passed through the mixed liquid L1 in the tube TB (light having passed through the fifth lens 47). In the first embodiment, the detection unit 48 includes a photodiode, and outputs a voltage corresponding to an amount of received light to the control unit 6. In the detection unit 48, light having the wavelength λ1 and emitted from the first light source and light having the wavelength λ2 and emitted from the second light source 49 are detected, and the control unit 6 calculates absorbance on the basis of a voltage corresponding to each detected light reception amount. In this way, the absorbance measurement unit 4 measures absorbance in a plurality of different wavelength regions for the mixed liquid L1 in which different liquids are mixed.

### [Configuration of Control Unit]

The control unit 6 includes a controller such as a central processing unit (CPU) or a micro processing unit (MPU), or an integrated circuit such as an application specific integrated circuit (ASIC) or a field programmable gate array (FPGA). Then, the control unit 6 calculates absorbance on the basis of a voltage corresponding to a received light amount detected by the detection unit 48. The control unit 6 corrects a concentration of at least one liquid on the basis of absorbance in the plurality of different wavelength regions measured by the absorbance measurement unit 4. Furthermore, the control unit 6 can calculate a mixing ratio of a liquid mixed in the mixed liquid, from absorbance in the plurality of different wavelength regions. Furthermore, the control unit 6 can also correct absorbance on the basis of the mixing ratio of the liquid mixed in the mixed liquid. Moreover, the control unit 6 adjusts a concentration of the mixed liquid L1 by calculating the concentration in the mixed liquid L1 on the basis of a measurement result obtained by the absorbance measurement unit 4 and controlling operations of the pumps PO1 to PO3 and the valves V1 to V3 as described above.

### [Configuration of Calibration Curve Creation Unit]

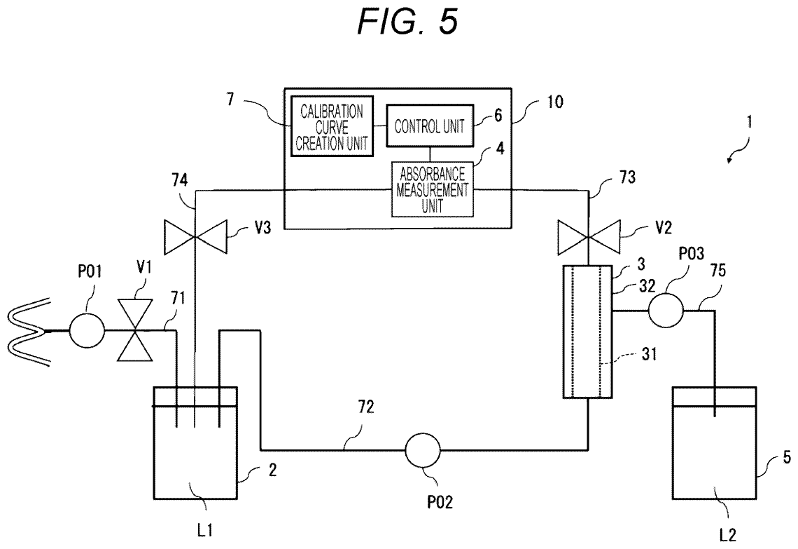

FIG. 5 is a diagram illustrating another configuration of the absorbance measurement unit in the concentration measurement device 10 according to the present embodiment. As illustrated in FIG. 5, the concentration measurement device 10 may further include the calibration curve creation unit 7.

The calibration curve creation unit 7 illustrated in FIG. 5 creates a calibration curve on the basis of absorbance calculated by the absorbance measurement unit 4 and the control unit 6. The calibration curve creation unit 7 creates a calibration curve by using a calibration-curve-creation mixed liquid in which a component is the same as a component of the mixed liquid L1 and a concentration of each mixed liquid is known.

The calibration curve creation unit 7 can create a calibration curve for obtaining a concentration of at least one mixed liquid, on the basis of absorbance in a specific wavelength region by using the calibration-curve-creation mixed liquid in which a concentration of the at least one liquid is known. Furthermore, the calibration curve creation unit 7 can create a calibration curve for obtaining a mixing ratio of a liquid mixed in the mixed liquid L1, on the basis of a difference in absorbance in the plurality of different wavelength regions. Moreover, the calibration curve creation unit 7 can create a calibration curve for obtaining absorbance on the basis of a mixing ratio of a liquid mixed in the mixed liquid L1.

### (3) Description of Concentration Calculation Method

A calculation method for a concentration of a mixed liquid in which different liquids are mixed performed by the control unit 6 will be described.

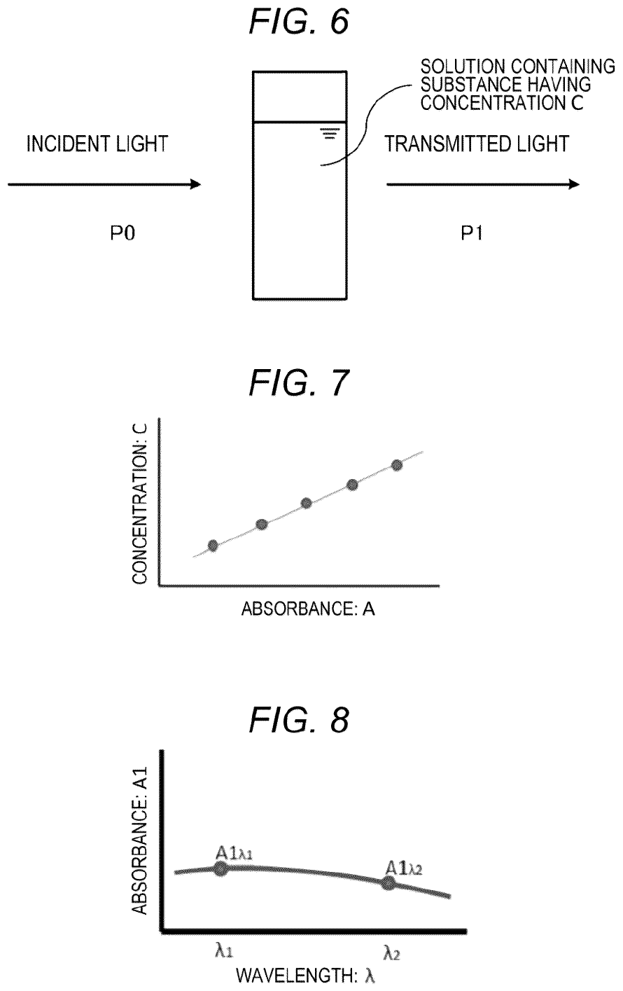

FIG. 6 is a diagram illustrating a calculation method for a concentration of a solution containing a substance having Concentration C.

It is known that absorbance of a solution is proportional to a concentration of the solution, according to Lambert-Beer's law.

### Lambert-Beer's Law

Absorbance (A)=εCLen
ε: Molar absorbance coefficient [L/(mol·cm)]
C: Concentration [mol/L]
Len: Optical path length [cm]

The concentration of the solution can be calculated by measuring the absorbance.

Furthermore, as illustrated in FIG. 6, the absorbance can be calculated on the basis of the following Equation (1) by measuring an incident light amount P0 and a transmitted light amount P1.

$$\begin{matrix}[ Formula 1 ] & \\ \begin{matrix}Absorbance ( A ) = - log ( T ) \\ = - log ( \frac{P 1}{P 0} )\end{matrix} & ( 1 )\end{matrix}$$

T: Transmissivity
P: Transmitted light amount
P0: Incident light amount

Meanwhile, the absorbance can be measured by Equation (1), but in this method, absorbance of the tube TB is also reflected in a measurement result. Therefore, the control unit 6 sets, to V0, a voltage detected by the detection unit 48 when a reference liquid having a concentration of 0 (not including particles) as a reference is put into the tube TB and measured, and calculates absorbance on the basis of the following Equation (2), where V is a voltage detected by the detection unit 48 when a liquid as a measurement target containing particles is put into the same tube TB and measured.

$$\begin{matrix}[ Formula 2 ] & \\ Absorbance ( A ) = log ( \frac{V 0}{V} ) & ( 2 )\end{matrix}$$

However, a liquid contained in a sample to be measured is not necessarily one type, and the sample to be measured may be, for example, a mixed liquid of two types of liquids. In this case, even if a relationship of Equation (1) described above is known for one liquid, the concentration cannot be correctly obtained from absorbance due to an influence of another liquid. Hereinafter, a calculation method for a concentration of a liquid contained in a mixed liquid will be described.

### [Creation of Calibration Curve (1)]

When calculating a concentration of a liquid contained in a mixed liquid, the calibration curve creation unit 7 creates Calibration curve (1) in advance for calculating the concentration from absorbance before measuring absorbance of a sample to be measured as a measurement target.

In a case of obtaining a concentration from absorbance of Solution 1, Calibration curve (1) for calculating the concentration from the absorbance is experimentally created. A plurality of samples of Solution 1 having different concentrations is prepared, and absorbance is measured in the absorbance measurement unit 4 at a wavelength λ2. FIG. 7 is a graph illustrating a relationship between Absorbance A and Concentration C. From the Lambert-Beer law, Absorbance A and Concentration C have a positive correlation, and thus a calibration curve in this case is expressed by Equation (3).

$$C = a \cdot A + b \qquad ( 3 )$$

C: Concentration
A: Absorbance
a, b: Constant

### [Creation of Calibration Curve (2)]

Before measuring absorbance of a sample to be measured, the calibration curve creation unit 7 creates Calibration curve (2) in advance for obtaining a mixing ratio from an absorbance difference of two wavelengths.

A case where Solution 1 and Solution 2 having different properties are mixed is considered.

Here, it is assumed that a difference between absorbance measured at the wavelength λ1 and absorbance measured at the wavelength λ2 different from the wavelength λ1 is small for Solution 1, and a difference between absorbance measured at the wavelength λ1 and absorbance measured at the wavelength λ2 is large for Solution 2. At this time, for Solution 1, it suffices that a difference between the absorbance measured at the wavelength λ1 and the absorbance measured at the wavelength λ2 is small, and a difference in absorbance does not need to be small in the entire wavelength range. Here, absorbance of each solution is defined as follows.
A1λ1: absorbance of Solution 1 at wavelength λ1
A1λ2: absorbance of Solution 1 at wavelength λ2
A2λ1: absorbance of Solution 2 at wavelength λ1
A2λ2: absorbance of Solution 2 at wavelength λ2
A12λ1: absorbance of mixed liquid of Solution 1 and Solution 2 at wavelength λ1
A12λ2: absorbance of mixed liquid of Solution 1 and Solution 2 at wavelength λ2

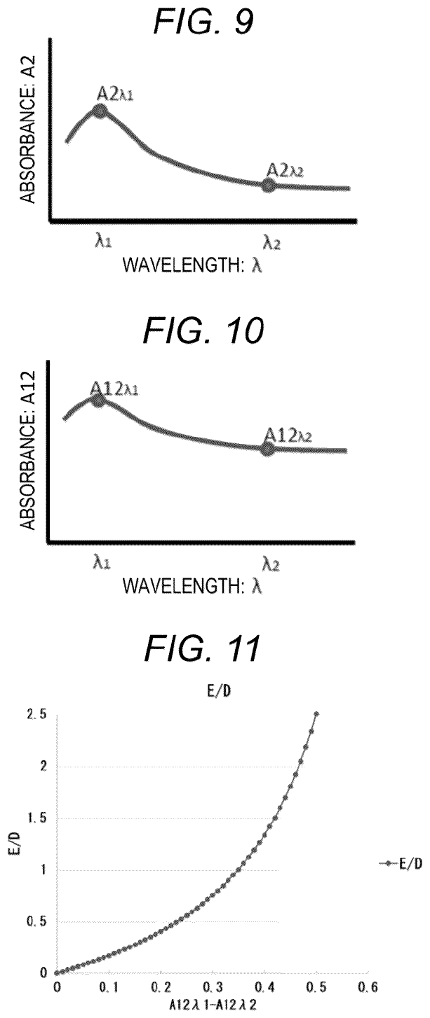

For example, it is assumed that an absorbance spectrum of Solution 1 has relatively flat characteristics as illustrated in FIG. 8, and an absorbance spectrum of Solution 2 has distinct characteristics as illustrated in FIG. 9. It is conceivable that, as Solution 2 is gradually added to Solution 1, an absorbance spectrum of the mixed solution becomes closer to the characteristics of Solution 2 as illustrated in FIG. 10. Therefore, as a rate of Solution 2 in the mixed liquid increases, a difference between Absorbance A12λ1 at the wavelength λ1 and Absorbance A12λ2 at the wavelength λ2 increases. Therefore, there is a correlation between “A12λ1−A12λ2” and “mixing ratio”. Since the absorbance has additivity, it suffices that a weighted average is used as the absorbance of the mixed solution obtained by mixing Solution 1 and Solution 2 at D: E, and the following Equations (4) and (5) are established.

$$\begin{matrix}[ Formula 3 ] & \\ A 12 λ1 = \frac{D \cdot A 1 λ1 + E \cdot A 2 λ1}{D + E} & ( 4 )\end{matrix}$$

$$\begin{matrix}[ Formula 4 ] & \\ A 12 λ2 = \frac{D \cdot A 1 λ2 + E \cdot A 2 λ2}{D + E} & ( 5 )\end{matrix}$$

When the absorbance difference (Equation (4)-Equation (5)) is calculated, the following equation is obtained.

$$A 12 λ1 - A 12 λ2 = \frac{D \cdot A 1 λ1 + E \cdot A 2 λ1}{D + E} - \frac{D \cdot A 1 λ2 + E \cdot A 2 λ2}{D + E}$$

When a numerator and a denominator on the right side are divided by D, the following equation is obtained.

$$\begin{matrix}[ Formula 6 ] & \\ A 12 λ 1 - A 12 λ 2 = \frac{A 1 λ 1 + E / D \cdot A 2 λ 1}{1 + E / D} - \frac{A 1 λ 2 + E / D \cdot A 2 λ 2}{1 + E / D} & \end{matrix}$$

When the above equation is solved for E/D, Calibration curve (2) expressed by the following Equation (6) can be obtained.

$$\begin{matrix}[ Formula 7 ] & \\ E / D = \frac{( A 1 λ 1 - A 1 λ 2 ) - ( A 12 λ 1 - A 12 λ 2 )}{( A 12 λ 1 - A 12 λ 2 ) - ( A 2 λ 1 - A 2 λ 2 )} & ( 6 )\end{matrix}$$

As expressed by Equation (6), if Absorbance A1λ1 and Absorbance A1λ2 of Solution 1 alone and Absorbance A2λ1 and Absorbance A2λ2 of Solution 2 alone are known in advance, the mixing ratio E/D can be obtained by measuring the absorbance difference between Absorbance At λ1 and Absorbance At λ2 of the mixed solution.

If each absorbance is set as follows, the graph of Equation (6) is as illustrated in FIG. 11.
A1λ1=0.5, A1λ2=0.5
A2λ1=1.4, A2λ2=0.7

However, in practice, since Solution 1 and Solution 2 are mixed in advance, it may be difficult to separately measure the absorbance of Solution 1 alone and the absorbance of Solution 2 alone. In this case, absorbance of a plurality of mixed liquids having a known mixing ratio may be measured in advance, and a calibration curve for obtaining the mixing ratio from the absorbance difference may be experimentally obtained.

### [Creation of Calibration Curve (3)]

Before measuring absorbance of a sample to be measured, the calibration curve creation unit 7 creates Calibration curve (3) for obtaining the absorbance from a mixing ratio in advance.

When a numerator and a denominator on the right side of the above Equation (5) are divided by D, Calibration curve (3) expressed by the following Equation (7) can be obtained.

$$\begin{matrix}[ Formula 8 ] & \\ A 12 λ 2 = \frac{A 1 λ 2 + E / D \cdot A 2 λ 2}{1 + E / D} & ( 7 )\end{matrix}$$

If A1λ2 and A2λ2 can be measured by Equation (7), the absorbance of the mixed liquid in which Solution 1 and Solution 2 are mixed at any mixing ratio E/D can be estimated.

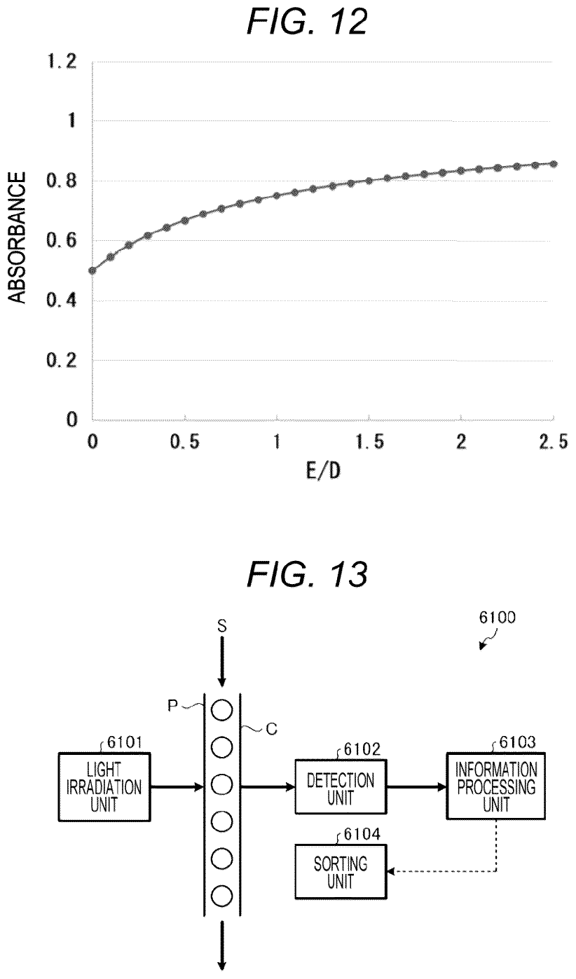

If each absorbance is set as follows, the graph of Equation (7) is as illustrated in FIG. 12.
A1λ2=0.5, A2λ2=1.0

However, in practice, Solution 1 and Solution 2 are mixed in advance, and it may be difficult to purify each of Solution 1 and Solution 2. In this case, a plurality of mixed liquids having a known mixing ratio may be measured in advance, and Calibration curve (3) for obtaining absorbance from the mixing ratio may be experimentally obtained.

### [Measurement Procedure]

The absorbance measurement unit 4 measures absorbance of a sample to be measured at two wavelengths, and the control unit 6 calculates a correction amount of the absorbance by using Calibration curve (2) and Calibration curve (3) created by the calibration curve creation unit 7, corrects the measured absorbance, and obtains a concentration of one Solution 1 by using Calibration curve (1).

The absorbance measurement unit 4 measures absorbance of a sample to be measured at two wavelengths (“s” is assigned in the sense of “sample to be measured”).
Absorbance at wavelength λ1: A12λ1s
Absorbance at wavelength λ2: A12λ2s

The control unit 6 calculates a mixing ratio E/Ds (“s” is assigned in the sense of “sample to be measured”) expressed by the following equation from Calibration curve (2).

$$\begin{matrix}[ Formula 9 ] & \\ E / Ds = \frac{( A 1 λ 1 - A 1 λ 2 ) - ( A 12 λ 1 s - A 12 λ 2 s )}{( A 12 λ 1 s - A 12 λ 2 s ) - ( A 2 λ 1 - A 2 λ 2 )} & \end{matrix}$$

Since Calibration curve (1) is a calibration curve at a certain mixing ratio E/D₍₁₎, it is necessary to correct the absorbance of the sample to be measured to absorbance of a case of the mixing ratio E/D₍₁₎, in order to obtain the concentration by using Calibration curve (1). Absorbance at any mixing ratio can be obtained by using Calibration curve (3), but A1λ2 and A2λ2 in Calibration curve (3) are absorbance at a certain concentration measured in advance, and are different from the concentration of the sample to be measured. Therefore, an absorbance difference due to the difference in mixing ratio is obtained on Calibration curve (3), and the absorbance difference is applied to Calibration curve (1) to correct the absorbance. That is, in Calibration curve (3), by obtaining absorbance at the mixing ratio E/Ds and absorbance at the mixing ratio E/D₍₁₎, and subtracting a difference Ad from actually measured absorbance, the absorbance can be corrected. Hereinafter, a method for obtaining the corrected concentration will be described.

First, the control unit 6 obtains Absorbance A(3)s at the mixing ratio E/Ds obtained by Calibration curve (2).

$$\begin{matrix}[ Formula 10 ] & \\ A ( 3 ) s = \frac{A 1 λ 2 + E / Ds \cdot A 2 λ 2}{1 + E / Ds} & \end{matrix}$$

Next, the control unit 6 obtains Absorbance A(3)₍₁₎ at the mixing ratio E/D₍₁₎ of the time of creating Calibration curve (1).

$$\begin{matrix}[ Formula 11 ] & \\ {A ( 3 )}_{( 1 )} = \frac{A 1 λ 2 + E / {D}_{( 1 )} \cdot A 2 λ 2}{1 + E / {D}_{( 1 )}} & \end{matrix}$$

Therefore, a correction amount Ad of the absorbance is expressed by the following equation.

$$Ad = A ( 3 ) s = {A ( 3 )}_{( 1 )}$$

Since the absorbance (measured value) of the mixed liquid before correction is A12λ2, the corrected absorbance Ac is expressed by the following equation.

$$Ac = A 12 λ 2 - Ad$$

By substituting the corrected absorbance Ac into Calibration curve (1), a corrected concentration Cc can be calculated as shown by the following equation.

$$Cc = a \cdot Ac + b$$

### (4) Configuration of Specimen Analyzer

Next, a configuration of the specimen analyzer 6100 will be described.

A configuration example of the specimen analyzer 6100 is illustrated in FIG. 13. The specimen analyzer 6100 illustrated in FIG. 13 includes a light irradiation unit 6101 that irradiates a specimen S flowing through a flow channel C with light, a detection unit 6102 that detects light generated by irradiating the specimen S with light, and an information processing unit 6103 that processes information regarding the light detected by the detection unit 6102. The specimen analyzer 6100 is a flow cytometer or an imaging cytometer, for example. The specimen analyzer 6100 may include a sorting unit 6104 that sorts specific particles P in the specimen. The specimen analyzer 6100 including the sorting unit 6104 is a cell sorter, for example.

### [Specimen]

The specimen S is a mixed liquid containing different liquids. Each of the different liquids may be a liquid specimen containing particles, or a liquid specimen containing biological particles. Furthermore, particles contained in one of the different liquids may be different from particles contained in another of the different liquids. The biological particles are cells or non-cellular biological particles, for example. The cells may be living cells, and more specific examples thereof include blood cells such as red blood cells and white blood cells, and germ cells such as sperms and fertilized eggs. The mixed liquid may be a suspension or a solution, and may contain a white blood cell suspension and a red blood cell suspension. Furthermore, the cells may be those directly collected from a sample such as whole blood, or may be cultured cells obtained after culturing. The non-cellular biological particles are extracellular vesicles, or particularly, exosomes and microvesicles, for example. The biological particles may be labeled with one or more labeling substances (for example, (particularly, a fluorescent dye) and a fluorochrome-labeled antibody). Note that particles other than biological particles may be analyzed by the biological specimen analyzer of the present disclosure, and beads or the like may be analyzed for calibration or the like.

### [Flow Channel]

The flow channel C is configured such that the specimen S flows. In particular, the flow channel C can be configured such that a flow is formed in which particles contained in the specimen S are aligned in a substantially one row. The flow channel structure including the flow channel C may be designed so that a laminar flow is formed. In particular, the flow channel structure is designed so that a laminar flow in which the flow of the specimen S (a sample flow) is surrounded by the flow of a sheath liquid is formed. The design of the flow channel structure may be appropriately selected by a person skilled in the art, or a known one may be adopted. The flow channel C may be formed in a flow channel structure such as a microchip (a chip having a flow channel on the order of micrometers) or a flow cell. The width of the flow channel C is 1 mm or smaller, or particularly, may be not smaller than 10 μm and not greater than 1 mm. The flow channel C and the flow channel structure including the flow channel C may be made containing a material such as plastic or glass.

The specimen analyzer is configured such that the specimen S flowing in the flow channel C, particularly particles in the specimen S, are irradiated with light from the light irradiation unit 6101. The specimen analyzer may be designed such that an irradiation point (interrogation point) of light on the specimen S is located in a flow channel structure in which the flow channel C is formed, or may be designed such that the irradiation point is located outside the flow channel structure. An example of the former case may be a configuration in which the light is emitted onto the flow channel C in a microchip or a flow cell. In the latter case, the particles after exiting the flow channel structure (particularly, a nozzle portion thereof) may be irradiated with the light, and a flow cytometer of a jet-in-air type can be adopted, for example.

### [Light Irradiation Unit]

The light irradiation unit 6101 includes a light source unit (not illustrated) that emits light and a light guide optical system (not illustrated) that guides the light to an irradiation point. The light source unit includes one or more light sources. The type of the light source(s) is a laser light source or an LED, for example. The wavelength of light to be emitted from each light source may be any wavelength of ultraviolet light, visible light, and infrared light. The light guide optical system includes optical components such as beam splitters, mirrors, or optical fibers, for example. The light guide optical system may also include a lens group for condensing light, and includes an objective lens, for example. The number of the irradiation points at which the specimen S and the light intersect may be one or more. The light irradiation unit 6101 may be designed to collect light emitted onto one irradiation point from one light source or different light sources.

### [Detection Unit]

The detection unit 6102 includes at least one photodetector (not illustrated) that detects light generated by irradiating particles with light. The light to be detected may be fluorescence or scattered light (such as one or more of the following: forward scattered light, backscattered light, and side scattered light), for example. Each photodetector includes one or more light receiving elements, and has a light receiving element array, for example. Each photodetector may include one or more photomultiplier tubes (PMTs) and/or photodiodes such as APDs and MPPCs, as the light receiving elements. The photodetector includes a PMT array in which a plurality of PMTs is arranged in a one-dimensional direction, for example. The detection unit 6102 may also include an image sensor such as a CCD or a CMOS. With the image sensor, the detection unit 6102 can acquire an image (such as a bright-field image, a dark-field image, or a fluorescent image, for example) of particles.

The detection unit 6102 includes a detection optical system (not illustrated) that causes light having a predetermined detection wavelength to reach a corresponding photodetector. The detection optical system includes a spectroscopic unit such as a prism or a diffraction grating, or a wavelength separation unit such as a dichroic mirror or an optical filter. The detection optical system is designed to disperse the light generated by light irradiation to particles, for example, and detect the dispersed light with a larger number of photodetectors than the number of fluorescent dyes with which the particles are labeled. A flow cytometer including such a detection optical system is called a spectral flow cytometer. Furthermore, the detection optical system is designed to separate the light corresponding to the fluorescence wavelength band of a specific fluorescent dye from the light generated by the light irradiation to the particles, for example, and cause the corresponding photodetector to detect the separated light.

Furthermore, the detection unit 6102 can include a signal processing unit (not illustrated) that converts an electric signal obtained by the photodetector into a digital signal. The signal processing unit may include an A/D converter as a device that performs the conversion. The digital signal obtained by the conversion performed by the signal processing unit can be transmitted to the information processing unit 6103. The digital signal can be handled as data related to light (hereinafter, also referred to as “light data”) by the information processing unit 6103. The light data may be light data including fluorescence data, for example. More specifically, the light data may be data of light intensity, and the light intensity may be light intensity data of light including fluorescence (the light intensity data may include feature quantities such as area, height, and width).

### [Information Processing Unit]

The information processing unit 6103 includes, for example, a processing unit (not illustrated) that executes processing on various kinds of data (for example, light data) and a storage unit (not illustrated) that stores various kinds of data. In a case where the processing unit acquires the light data corresponding to a fluorescent dye from the detection unit 6102, the processing unit can perform fluorescence leakage correction (a compensation process) on the light intensity data. In the case of a spectral flow cytometer, the processing unit also performs a fluorescence separation process on the light data, and acquires the light intensity data corresponding to the fluorescent dye. The fluorescence separation process may be performed by an unmixing method disclosed in JP 2011-232259 A, for example. In a case where the detection unit 6102 includes an image sensor, the processing unit may acquire morphological information about the particles, on the basis of an image acquired by the image sensor. The storage unit may be designed to be capable of storing the acquired light data. The storage unit may be designed to be capable of further storing spectral reference data to be used in the unmixing process.

In a case where the specimen analyzer 6100 includes the sorting unit 6104 described later, the information processing unit 6103 can determine whether to sort the particles, on the basis of the light data and/or the morphological information. Then, the information processing unit 6103 then controls the sorting unit 6104 on the basis of the result of the determination, and the particles can be sorted by the sorting unit 6104.

The information processing unit 6103 may be designed to be capable of outputting various kinds of data (such as light data and images, for example). For example, the information processing unit 6103 can output various kinds of data (such as a two-dimensional plot or a spectrum plot, for example) generated on the basis of the light data. The information processing unit 6103 may also be designed to be capable of accepting inputs of various kinds of data, and accepts a gating process on a plot by a user, for example. The information processing unit 6103 can include an output unit (such as a display, for example) or an input unit (such as a keyboard, for example) for performing the output or the input.

The information processing unit 6103 may be designed as a general-purpose computer, and may be designed as an information processing device that includes a CPU, a RAM, and a ROM, for example. The information processing unit 6103 may be included in the housing in which the light irradiation unit 6101 and the detection unit 6102 are included, or may be located outside the housing. Furthermore, the various processes or functions to be executed by the information processing unit 6103 may be realized by a server computer or a cloud connected via a network.

### [Sorting Unit]

The sorting unit 6104 performs sorting of particles, in accordance with the result of determination performed by the information processing unit 6103. The sorting method may be a method by which droplets containing particles are generated by vibration, electric charges are applied to the droplets to be sorted, and the traveling direction of the droplets is controlled by an electrode. The sorting method may be a method for sorting by controlling the traveling direction of particles in the flow channel structure. The flow channel structure has a control mechanism based on pressure (injection or suction) or electric charge, for example. An example of the flow channel structure may be a chip (the chip disclosed in JP 2020-76736 A, for example) that has a flow channel structure in which the flow channel C branches into a recovery flow channel and a waste liquid flow channel on the downstream side, and specific particles are collected in the recovery flow channel.

### OTHER EMBODIMENTS

Although the embodiment for carrying out the present technology has been described so far, the present technology should not be limited only by the above-described first embodiment.

The above-described configuration of the concentration adjustment device 1 is merely an example, and other configurations may be adopted.

### [Hardware Configuration]

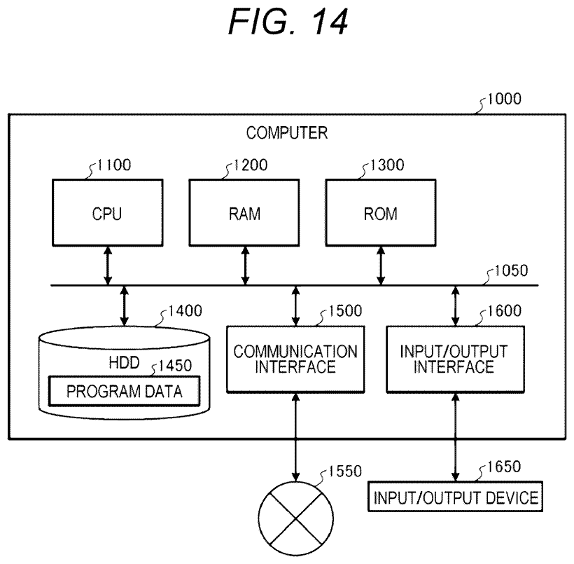

The control unit 6 according to the above-described embodiment can be implemented by a computer 1000 having a configuration as illustrated in FIG. 14, for example. FIG. 14 is a hardware configuration diagram illustrating an example of the computer 1000 that implements the functions of the control unit 6. The computer 1000 includes a CPU 1100, a RAM 1200, a read only memory (ROM) 1300, a hard disk drive (HDD) 1400, a communication interface 1500, and an input/output interface 1600. Each unit of the computer 1000 is connected by a bus 1050.

The CPU 1100 operates on the basis of a program stored in the ROM 1300 or the HDD 1400, and controls each unit. For example, the CPU 1100 develops the program stored in the ROM 1300 or the HDD 1400 in the RAM 1200, and executes processing corresponding to various programs.

The ROM 1300 stores a boot program such as a basic input output system (BIOS) executed by the CPU 1100 when the computer 1000 is activated, a program depending on hardware of the computer 1000, and the like.

The HDD 1400 is a computer-readable recording medium that non-transiently records the program executed by the CPU 1100, data used by the program and the like. Specifically, the HDD 1400 is a recording medium that records a program for executing each operation according to the present technology, which is an example of program data 1450.

The communication interface 1500 is an interface for the computer 1000 to connect to an external network 1550 (for example, the Internet). For example, the CPU 1100 receives data from another device or transmits data generated by the CPU 1100 to another device via the communication interface 1500.

The input/output interface 1600 is an interface for connecting an input/output device 1650 to the computer 1000. For example, the CPU 1100 receives data from an input device such as a keyboard and a mouse via the input/output interface 1600. Furthermore, the CPU 1100 transmits data to an output device such as a display, a speaker, and a printer via the input/output interface 1600. Furthermore, the input/output interface 1600 may serve as a media interface that reads a program and the like recorded in a predetermined recording medium (medium). The medium is, for example, an optical recording medium such as a digital versatile disc (DVD) and a phase change rewritable disk (PD), a magneto-optical recording medium such as a magneto-optical disk (MO), a tape medium, a magnetic recording medium, a semiconductor memory, or the like.

For example, in a case where the computer 1000 functions as the control unit 6 according to the above-described embodiment, the CPU 1100 of the computer 1000 implements the functions of the control unit 6 by executing a program loaded on the RAM 1200. Furthermore, the HDD 1400 stores a program and the like according to the present disclosure. Note that, although the CPU 1100 reads the program data 1450 from the HDD 1400 and executes the program data 1450, as another example, these programs may be acquired from another device via the external network 1550.

Note that the information processing unit 6103 constituting the specimen analyzer 6100 can also be implemented by a hardware configuration similar to that of the computer 1000 described above.

### 3. Second Embodiment (Example of Concentration Measurement Method)

### (1) Description of Concentration Measurement Method According to Present Embodiment

A concentration measurement method according to the present embodiment includes: an absorbance measuring step of measuring absorbance in a plurality of different wavelength regions for a mixed liquid in which different liquids are mixed; and a correction step of correcting a concentration of at least one liquid on the basis of absorbance in the plurality of different wavelength regions.

Furthermore, the concentration measurement method according to the present embodiment further includes: a calibration curve creation step of creating a calibration curve by using a calibration-curve-creation mixed liquid in which a component is same as a component of the mixed liquid and a concentration of each mixed liquid is known.

### (2) Description of Each Step

### [Absorbance Measuring Step]

The absorbance measuring step is a step of measuring absorbance in a plurality of different wavelength regions for a mixed liquid in which different liquids are mixed. Details of a measurement method performed in the absorbance measuring step are the same as the measurement method executed by the absorbance measurement unit 4 of the concentration measurement device 10 described above.

### [Correction Step]

In the correction step, a concentration of at least one solution is corrected on the basis of the absorbance in the plurality of different wavelength regions. Details of a correction method performed in the correction step are the same as the correction method executed by the control unit 6 of the concentration measurement device 10 described above.

### [Calibration Curve Creation Step]

In the calibration curve creating step, a calibration curve is created using a calibration-curve-creation mixed liquid in which a component is the same as a component of the mixed liquid and a concentration of each mixed liquid is known. Details of a calibration curve creating method performed in the calibration curve creating step are the same as the calibration curve creating method executed by the calibration curve creation unit 7 of the concentration measurement device 10 described above.

### 4. Examples

Hereinafter, the present technology will be described specifically with reference to Examples, but the present technology is not limited only to these Examples.

### Example 1

### [Creation of Calibration Curve (1)]

In order to measure a white blood cell concentration of a mixed liquid of Solution 1 that is a suspension of white blood cells (hereinafter, abbreviated as WBCs) and Solution 2 that is a suspension of red blood cells (hereinafter, abbreviated as RBCs), Calibration curve (1) is created in advance. In absorption characteristics, absorption of red blood cells is caused by hemoglobin (Hb) contained in the red blood cells. In a general absorption spectrum of hemoglobin (Hb), there is a characteristic peak in the vicinity of 420 nm, there is also a peak in the vicinity of 550 nm, and the absorbance decreases after 600 nm.

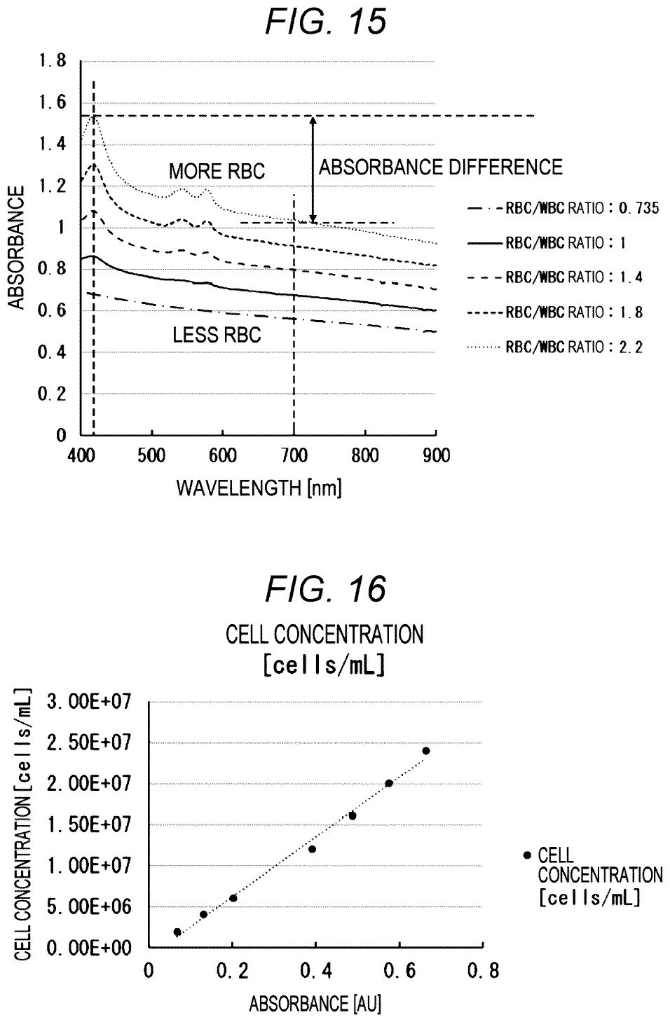

Furthermore, FIG. 15 shows a result of measuring an absorbance spectrum with the absorbance measurement unit 4, by thawing commercially available frozen PBMCs and adding whole blood little by little to adjust an RBC/WBC ratio. Note that the peripheral blood mononuclear cells (PBMCs) refer to mononuclear cells including monocytes and lymphocytes separated from peripheral blood. When a rate of RBCs increases, absorbance around 420 nm becomes relatively larger than absorbance at other wavelengths. For example, since a difference from absorbance around 700 nm increases, an RBC/WBC ratio of a sample to be measured can be estimated by calculating a difference between the absorbance at 420 nm and the absorbance at 700 nm.

Seven types of samples having different white blood cell (WBC) concentrations were prepared using commercially available PBMCs, and the absorbance of each sample was measured. Similar measurement was performed three times in total using PBMCs of another donor. An average value of the measured absorbance was obtained, the graph illustrated in FIG. 16 was created, and an approximate straight line was created to obtain Calibration curve (1) expressed by the following Equation (8). A wavelength used in the absorbance measurement was 700 nm, and an average value of the RBC/WBC ratio was 1.14.

$$C [ cell / mL ] = 3.64 \times {10}^{7} \times absorbance - 1.07 \times {10}^{6} \qquad ( 8 )$$

C: WBC concentration

### [Creation of Calibration Curve (2)]

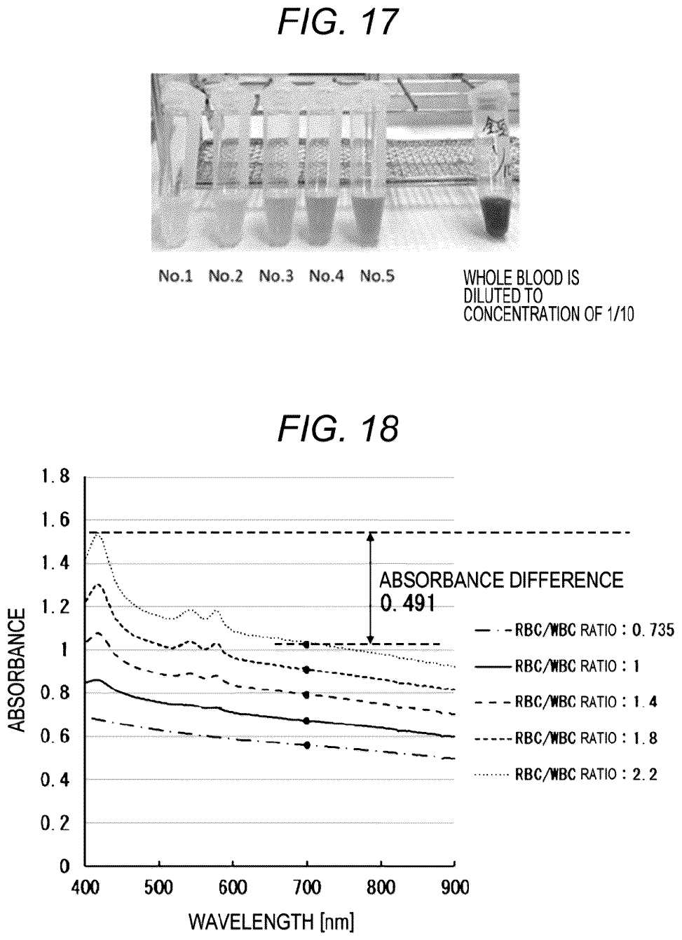

Next, Calibration curve (2) for obtaining an RBC/WBC ratio from an absorbance difference between two wavelengths is created. Since it is difficult to adjust a sample of only RBCs and a sample of only WBCs, the calibration curve was experimentally obtained as described above. Although the RBC/WBC ratio of commercially available PBMCs is around 1, as illustrated in FIG. 17, the RBC/WBC ratio of PBMCs was adjusted by adding whole blood diluted to a concentration of 1/10 (RBC/WBC ratio around 1000), and five types (Samples No. 1 to No. 5) of measurement samples having different RBC concentrations were prepared as shown in Table 1.

TABLE 1

| | | RBC | Amount of |
|---|---|---|---|
| | | concentration | added blood |
| Sample No. | RBC/WBC ratio | [cells/mL] | [μL] |
| 1 | 0.74 | 1.65E+07 | 0.00 |
| 2 | 1.0 | 2.23E+07 | 11.3 |
| 3 | 1.4 | 3.07E+07 | 28.3 |
| 4 | 1.8 | 3.88E+07 | 45.4 |
| 5 | 2.2 | 4.67E+07 | 62.4 |

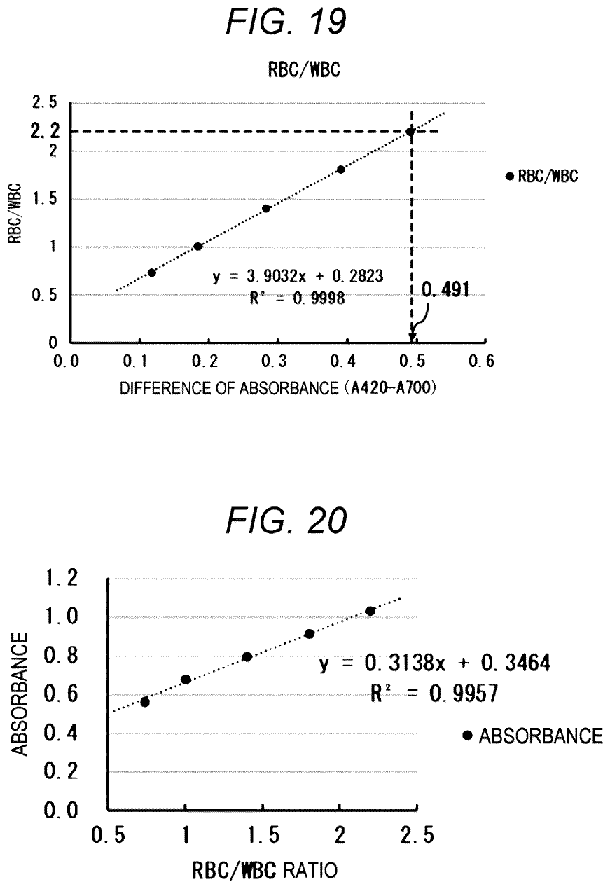

FIG. 18 illustrates an absorbance spectrum measured with a spectrophotometer. In measuring absorbance, 420 nm showing the characteristics of hemoglobin (Hb) most clearly and 700 nm that is relatively stable were selected as different wavelength regions. FIG. 19 is a graph in which a difference between the absorbance measured at 420 nm and the absorbance measured at 700 nm at each RBC/WBC ratio is obtained from FIG. 18, the absorbance difference is taken on a horizontal axis, and the RBC/WBC ratio is taken on a vertical axis. In FIG. 19, since R² was 0.9998 even when linear approximation was performed, it was determined that linear approximation was possible, and Calibration curve (2) expressed by the following Equation (9) was obtained by linear approximation.

$$RBC / WBC ratio = 3.9 \times absorbance difference + 0.282 \qquad ( 9 )$$

### [Creation of Calibration Curve (3)]

Finally, Calibration curve (3) for obtaining absorbance from the RBC/WBC ratio is created. Also in this case, since it was difficult to adjust a sample of only RBCs and a sample of only WBCs, Calibration curve (3) was experimentally obtained. FIG. 20 is a graph in which the absorbance at 700 nm in FIG. 18 is extracted, the RBC/WBC ratio is taken on a horizontal axis, and the absorbance is taken on a vertical axis. In FIG. 20, since R² was 0.9957 even when linear approximation was performed, it was determined that linear approximation was possible, and Calibration curve (3) expressed by the following Equation (10) was obtained by linear approximation.

$$Absorbance = 0.314 \cdot RBC / WBC ratio + 0.346 \qquad ( 10 )$$

### [Measurement of Sample to be Measured]

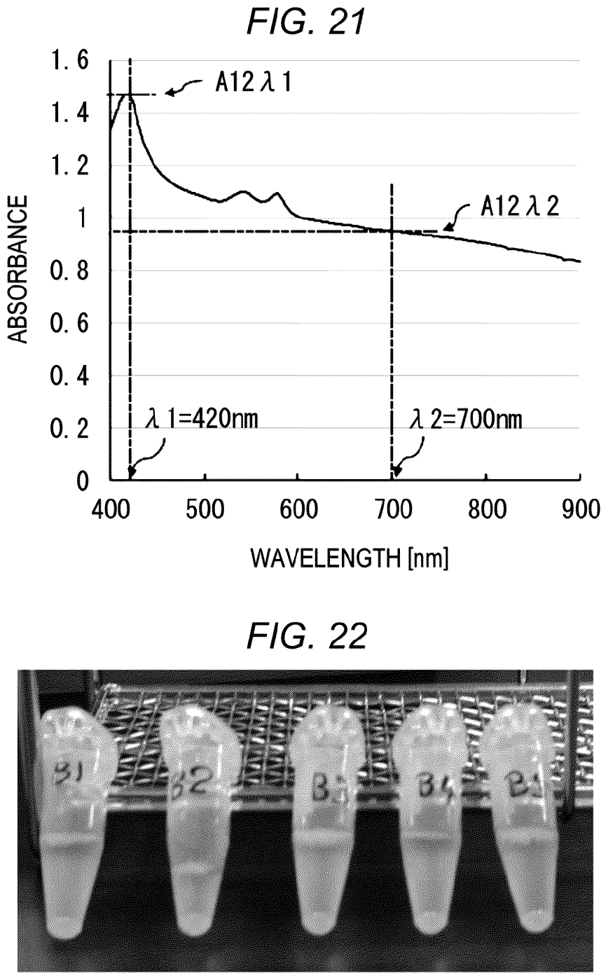

An absorbance spectrum measured using a specimen of a donor different from that of the time of creating the calibration curve is illustrated in FIG. 21. Absorbance A12λ1 at the wavelength λ1 (420 nm) and absorbance A12λ2 at the wavelength λ2 (700 nm) were extracted, and A12λ1−A12λ2 was calculated as expressed by the following Equation (11).

Note that the measurement may not be in a spectrum.

$$\begin{matrix}A 12 λ 1 = 1.47 \\ A 12 λ 2 = 0.951 \\ A 12 λ 1 - A 12 λ 2 = 0.519\end{matrix} \qquad ( 11 )$$

The absorbance difference 0.519 obtained by Equation (11) was substituted into Calibration curve (2) expressed by Equation (9) to calculate the RBC/WBC ratio.

$$\begin{matrix}RBC / WBC ratio = 3.9 \times absorbance difference + 0.282 \\ = 3.9 \times 0.519 + 0.282 \\ = 2.31\end{matrix}$$

Therefore, the RBC/WBC ratio of the sample to be measured was 2.31.

Next, absorbance when the RBC/WBC ratio of the sample to be measured was 2.31 and absorbance when the RBC/WBC ratio at the time of creating Calibration curve (1) was 1.14 were calculated using Calibration curve (3), and an absorbance difference therebetween was obtained.

According to Calibration curve (3) expressed by Equation (10), Absorbance As when the RBC/WBC ratio was 2.31 was 1.07 as expressed by the following equation.

$$\begin{matrix}As = 0.314 \times 2.31 + 0.346 \\ = 1.07\end{matrix}$$

According to Calibration curve (3) expressed by Equation (10), Absorbance Ac when the RBC/WBC ratio was 1.14 was 0.704 as expressed by the following equation.

$$\begin{matrix}Ac = 0.314 \times 1.14 ++ 0.346 \\ = 0.704\end{matrix}$$

Absorbance difference Ad was 0.366 as expressed by the following equation.

$$\begin{matrix}Ad = As - Ac \\ = 1.07 - 0.704 = 0.366\end{matrix}$$

Since the absorbance (measured value) of the mixed liquid before correction is A12λ2, the corrected absorbance Ac is expressed by the following equation.

$$A c = A 12 λ2 - Ad$$

Therefore, the corrected absorbance Ac when the RBC/WBC ratio of the sample to be measured was 1.14 was 0.585 as expressed by the following equation.

$$\begin{matrix}Ac = A 12 λ2 - Ad \\ = 0.951 - 0.366 \\ = 0.585\end{matrix}$$

By substituting the corrected absorbance Ac into Calibration curve (1), a corrected white blood cell concentration Cc can be calculated as shown by the following equation.

$$\begin{matrix}Cc [ cell / mL ] = 3.64 \times {10}^{7} \times absorbance - 1.07 \times {10}^{6} \\ = 3.64 \times {10}^{7} \times 0.585 - 1.07 \times {10}^{6} \\ = 2.02 \times {10}^{7}\end{matrix}$$

### [Improvement of Measurement Error]

Absorbance was measured using a specimen of a donor different from that of the time of creating the calibration curve. On the basis of the result, a white blood cell (WBC) concentration was calculated for each of a case where the absorbance was corrected and a case where the absorbance was not corrected. Furthermore, the white blood cell (WBC) concentration was measured using a commercially available automatic hemacytometer, and an error when this WBC concentration was regarded as positive was also calculated. The results thereof are shown in Table 2. When the errors are compared between a case with absorbance correction and a case without absorbance correction (conventional method), it is found that the measurement error is significantly improved by performing absorbance correction.

TABLE 2

| | Sample No. | | | | |
|---|---|---|---|---|---|
| | Sample 5 | Sample 4 | Sample 3 | Sample 2 | Sample 1 |
| WBC concentration [cells/mL] measured | 2.11E+07 | 2.15E+07 | 2.20E+07 | 2.24E+07 | 2.26E+07 |
| by automatic hemacytometer | | | | | |
| RBC/WBC ratio measured by automatic | 2.2 | 1.8 | 1.4 | 1 | 0.83 |
| hemacytometer | | | | | |
| Absorbance A12λ1 measured at wavelength | 1.470 | 1.268 | 0.963 | 0.780 | 0.680 |
| 420 nm | | | | | |
| Absorbance A12λ2 measured at wavelength | 0.951 | 0.854 | 0.703 | 0.606 | 0.547 |
| 700 nm | | | | | |
| Difference of absorbance (A12λ1 − | 0.519 | 0.414 | 0.259 | 0.174 | 0.133 |
| A12λ2) | | | | | |
| RBC/WBC ratio calculated with | 2.31 | 1.90 | 1.29 | 0.96 | 0.80 |
| Calibration curve (2) | | | | | |
| Correction amount of absorbance | −0.367 | −0.238 | −0.048 | 0.056 | 0.106 |
| calculated with Calibration curve (3) | | | | | |
| Absorbance in Calibration curve (1) | 0.584 | 0.616 | 0.655 | 0.661 | 0.653 |
| WBC concentration [cells/mL] calculated | 2.02E+07 | 2.14E+07 | 2.28E+07 | 2.30E+07 | 2.27E+07 |
| with Calibration curve (1) | | | | | |
| With absorbance correction | | | | | |
| Error | −4.24% | −0.86% | 3.64% | 2.61% | 0.39% |
| WBC concentration [cells/mL] calculated | 3.35E+07 | 3.00E+07 | 2.45E+07 | 2.10E+07 | 1.88E+07 |
| with Calibration curve (1) | | | | | |
| Without absorbance correction | | | | | |
| (conventional method) | | | | | |
| Error | 59.0% | 39.3% | 11.7% | −6.42% | −16.7% |

### Example 2

### [Creation of Calibration Curve (1)]

As a means for measuring a blood cell composition of a blood sample, there is an automatic hemacytometer, and RBCs are measured by a sheath flow DC detection method. In this method, cells are aligned in one row in the sheath flow and then caused to pass through the detection unit, a size of the cells is measured by detecting a change in electric resistance of the detection unit, and it is determined from the size that the cells are RBCs. For some reason, there is an RBC ghost in which a membrane is broken and hemoglobin (HGB) in the RBC leaks out, and only the membrane is present. Since the RBC ghost does not contain hemoglobin (HGB), the RBC ghost is almost transparent. In the sheath flow DC detection method, whether or not the cells are RBCs is determined by the size, and it cannot be determined whether or not HGB is included in RBCs. Therefore, the RBC ghost not including HGB and having low absorbance is also counted. Therefore, even with the same RBC contamination rate, there are a sample having high absorbance and a sample having low absorbance depending on a sample to be measured, and it is difficult to accurately correct the result of the in-line cell concentration measurement based on the number of RBCs. Whereas, HGB is detected by a non-cyanide HGB measurement method, and an amount of HGB is quantified by hemolyzing RBCs with a hemolytic reagent in an automatic hemacytometer, and measuring absorbance after all HGB has leaked from RBCs. Since HGB is measured by the same absorbance method as the in-line cell concentration measurement, it is possible to perform correction with higher accuracy than the RBC basis by using an HGB amount as a reference.

In order to measure a white blood cell concentration of a mixed liquid of Solution 1 that is a suspension of white blood cells (hereinafter, abbreviated as WBCs) and Solution 2 that is a suspension of hemoglobin (hereinafter, abbreviated as HGB), Calibration curve (1) is created in advance. In absorption characteristics, absorption of red blood cells is caused by hemoglobin (Hb) contained in the red blood cells. In a general absorption spectrum of hemoglobin (Hb), there is a characteristic peak in the vicinity of 420 nm, there is also a peak in the vicinity of 550 nm, and the absorbance decreases after 600 nm.

Commercially available frozen PBMCs were thawed, and whole blood was added little by little to adjust an HGB/WBC ratio, thereby preparing five types of samples with different WBC concentrations around 2×10⁷ cells/mL, which was a target concentration of CSC, as shown in Table 3 below. FIG. 22 is a photograph of each sample. The RBC/WBC ratio at this time was 0.436, and HGB/WBC was 25.8 pg/cells.

TABLE 3

| | WBC concentration (actual |
|---|---|
| | measurement value) |
| Sample No. | [cells/mL] |
| 1 | 7.72E+06 |
| 2 | 1.22E+07 |
| 3 | 1.57E+07 |
| 4 | 2.17E+07 |
| 5 | 2.38E+07 |

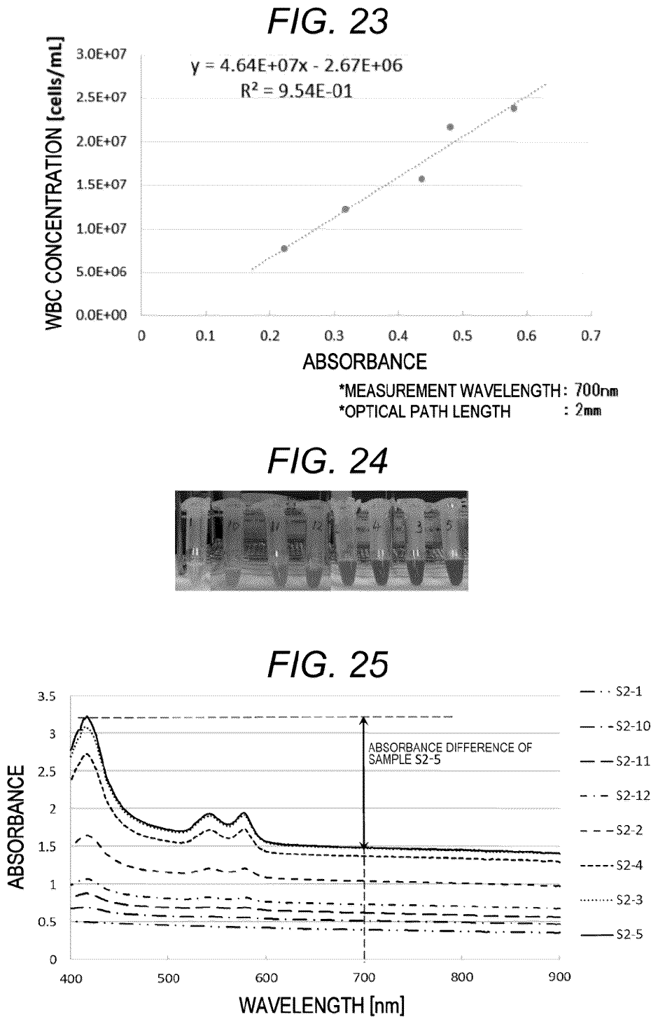

Next, absorbance of these was measured with a double beam spectrophotometer, and results of plotting the absorbance at 700 nm on a horizontal axis and the WBC concentration on a vertical axis are illustrated in FIG. 23. Using a function of Excel (registered trademark) which is software available from Microsoft Corporation of the United States, an approximate straight line expressed by the following Equation (12) was obtained and used as Calibration curve (1).

$$W B C concentration : C [ cells / mL ] = 4.64 \times {10}^{7} \times absorbance - 2.67 \times {10}^{6} \qquad ( 12 )$$

Calibration curve (1) is a basic calibration curve, and a correction amount of absorbance was calculated on the RBC basis or the HGB basis using Calibration curve (2) and Calibration curve (3) described later, and then substituted into Calibration curve (1).

### [Creation of Calibration Curve (2)]

Frozen PBMCs were thawed, adjusted to a concentration close to 2×10⁷ cells/mL, which is about a target concentration of CSC, and dispensed into eight tubes, and then a trace amount of whole blood was added to each sample to prepare samples having different RBC concentrations. The RBC/WBC ratio and HGB/WBC of each sample are shown in Table 4. FIG. 24 is a photograph of each sample. The WBC concentration at this time was about 1.8×10⁷ cells/mL.

TABLE 4

| | | HGB/WBC |
|---|---|---|
| Sample No. | RBC/WBC ratio | [pg/cells] |
| 1 | 0.79 | 2.44E+01 |
| 10 | 1.83 | 4.43E+01 |
| 11 | 2.18 | 5.84E+01 |
| 12 | 2.49 | 6.79E+01 |
| 2 | 2.90 | 9.45E+01 |
| 4 | 5.08 | 1.61E+02 |
| 3 | 6.03 | 1.90E+02 |
| 5 | 6.63 | 2.04E+02 |

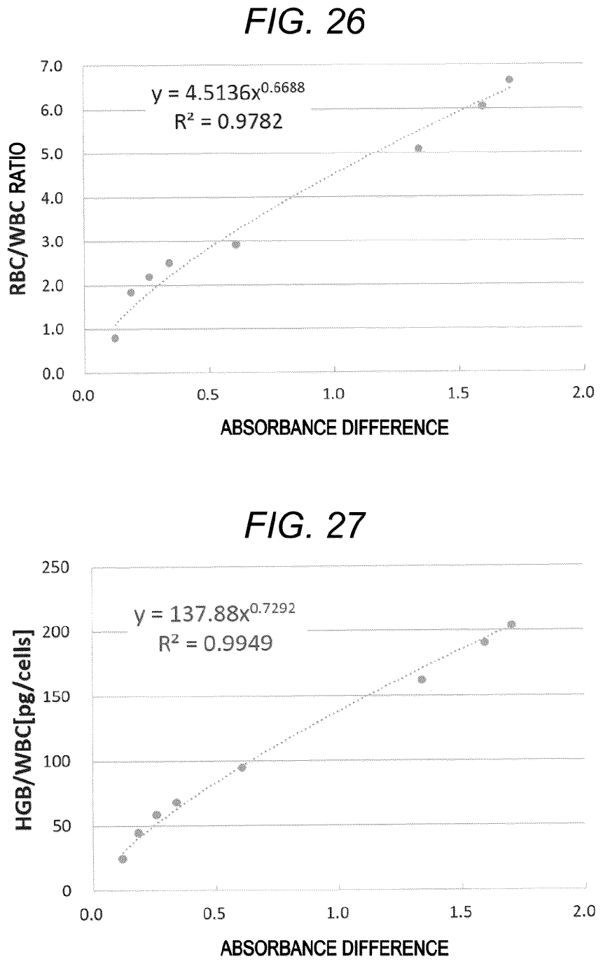

The results of measuring absorbance spectra of these samples with a double beam spectrophotometer are illustrated in FIG. 25. From the measurement results, an absorbance difference between 420 nm and 700 nm in each sample was obtained, a graph with the absorbance difference on a horizontal axis and the RBC/WBC ratio on a vertical axis is illustrated in FIG. 26, and a graph with the absorbance difference on a horizontal axis and HGB/WBC [pg/cells] on a vertical axis is illustrated in FIG. 27. Note that, in order to keep the WBC concentration constant and suppress an addition amount to be small, whole blood was added to each sample as it was (RBC/WBC ratio: 2081). Since the amount of whole blood to be added was as small as several μl, the RBC/WBC ratio of each sample was a ratio as actually obtained, although measures were taken such as using a low adsorption pipette tip (H-104-96RS-LR-Q of QSP). Therefore, the sample numbers are not in order.

A power approximate curve was obtained from FIG. 26 by using a function of Excel (registered trademark) which is software of Microsoft Corporation of the United States, and Calibration curve (2) of the RBC basis expressed by the following Equation (13) was obtained.

$$R B C / W B C ratio = 4.51 \times absorbance {difference}^{0.669} \qquad ( 13 )$$

Furthermore, a power approximate curve was obtained from FIG. 27 by using a function of Excel (registered trademark) which is software of Microsoft Corporation of the United States, and Calibration curve (2) of the HGB basis expressed by the following Equation (14) was obtained.

$$H G B / W B C [ pg / cells ] = 138 \times absorbance {difference}^{0.729} \qquad ( 14 )$$

### [Creation of Calibration Curve (3)]

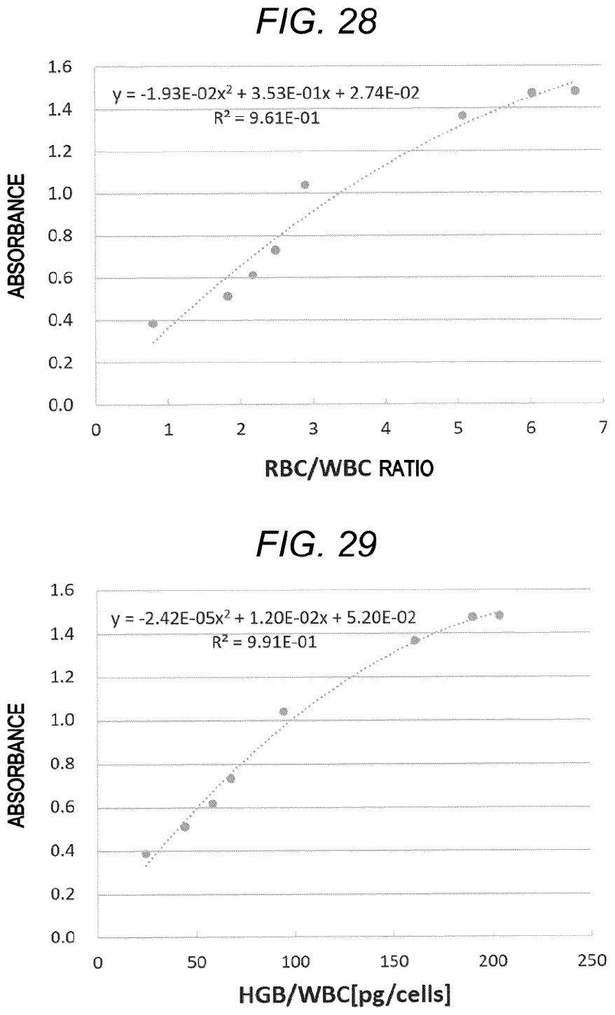

Absorbance of each sample at 700 nm was extracted from the measurement results in FIG. 25, and the RBC/WBC ratio and HGB/WBC [pg/cells] of each sample in Table 4 were referred to, a graph with the RBC/WBC ratio on a horizontal axis and absorbance on a vertical axis is illustrated in FIG. 28, and a graph with HGB/WBC [pg/cells] on a horizontal axis and absorbance on a vertical axis is illustrated in FIG. 29.

A polynomial approximate curve was obtained from FIG. 28 by using a function of Excel (registered trademark) which is software of Microsoft Corporation of the United States, and Calibration curve (3) of the RBC basis expressed by the following Equation (15) was obtained.

$$Absorbance = ( - 1.93 \times {10}^{- 2} ) \times {R}^{2} + ( 3.53 \times {10}^{- 1} ) \times R + ( 2.74 \times {10}^{- 2} ) \qquad ( 15 )$$

(R: RBC/WBC ratio)

A polynomial approximate curve was obtained from FIG. 29 by using a function of Excel (registered trademark) which is software of Microsoft Corporation of the United States, and Calibration curve (3) of the HGB basis expressed by the following Equation (16) was obtained.

$$Absorbance = ( - 2.42 \times {10}^{- 5} ) \times {H}^{2} + ( 1.2 \times {10}^{- 2} ) \times H + ( 5.2 \times {10}^{- 2} ) \qquad ( 16 )$$

(H: HGB/WBC [pg/cells])

### Example 3

### [Verification Experiment]

### (1) Sample Preparation

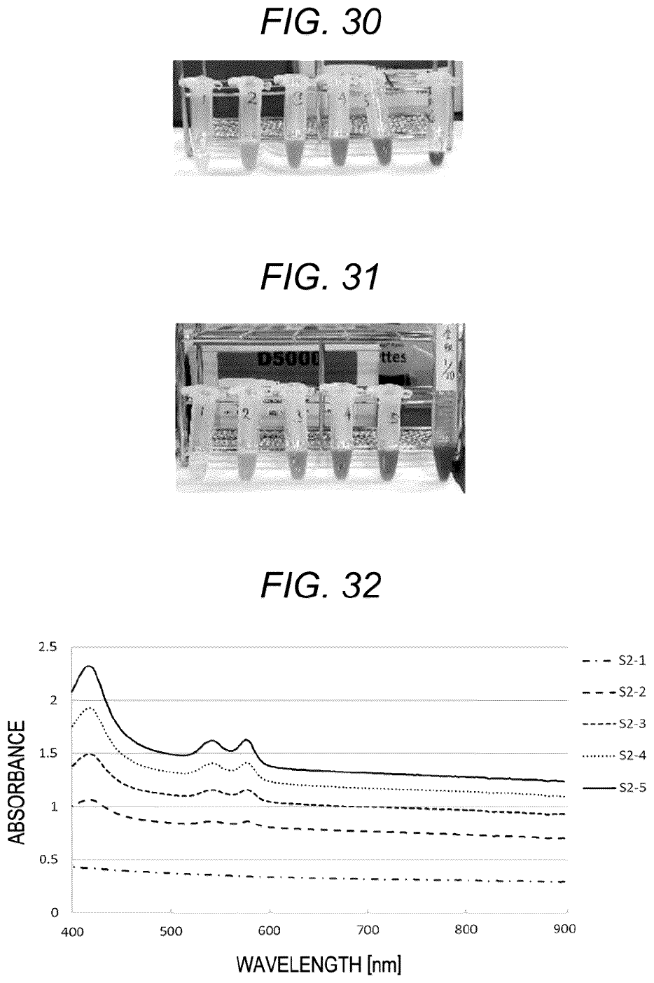

Similarly to Example 2, by using commercially available PBMCs, a small amount of whole blood was added to prepare five types of samples having different RBC/WBC ratios. Note that, since the whole blood had a high RBC concentration and it was difficult to finely adjust an amount of the added RBCs, the whole blood was diluted to 1/10 with PBS and then added to PBMCs. The RBC/WBC ratio, HGB/WBC, and the WBC concentration of each sample prepared for Verification experiment 1 are shown in Table 5. FIG. 30 is a photograph of each sample.

TABLE 5

| | | | WBC |
|---|---|---|---|
| | | HGB/WBC | concentration |
| Sample No. | RBC/WBC ratio | [pg/cells] | [cells/mL] |
| 1 | 0.56 | 26.9 | 1.79E+07 |
| 2 | 1.77 | 60.5 | 1.88E+07 |
| 3 | 2.34 | 87.4 | 1.75E+07 |
| 4 | 3.82 | 123 | 1.67E+07 |
| 5 | 4.40 | 149 | 1.68E+07 |

Similarly to Example 2, by using commercially available PBMCs, a small amount of whole blood of a donor different from the donor of Verification Experiment 1 was added to prepare five types of samples having different RBC/WBC ratios. Note that, since the whole blood had a high RBC concentration and it was difficult to finely adjust an amount of the added RBCs, the whole blood was diluted to 1/10 with PBS and then added to PBMCs. The RBC/WBC ratio, HGB/WBC, and the WBC concentration of each sample prepared for Verification experiment 2 are shown in Table 6. FIG. 31 is a photograph of each sample.

TABLE 6

| | | | WBC |
|---|---|---|---|
| | | HGB/WBC | concentration |
| Sample No. | RBC/WBC ratio | [pg/cells] | [cells/mL] |
| 1 | 0.58 | 26.0 | 2.03E+07 |
| 2 | 1.66 | 56.6 | 2.02E+07 |
| 3 | 2.23 | 85.7 | 1.96E+07 |
| 4 | 3.51 | 125 | 1.71E+07 |
| 5 | 4.68 | 162 | 1.66E+07 |

### (2) Measurement Result of Absorbance Spectrum

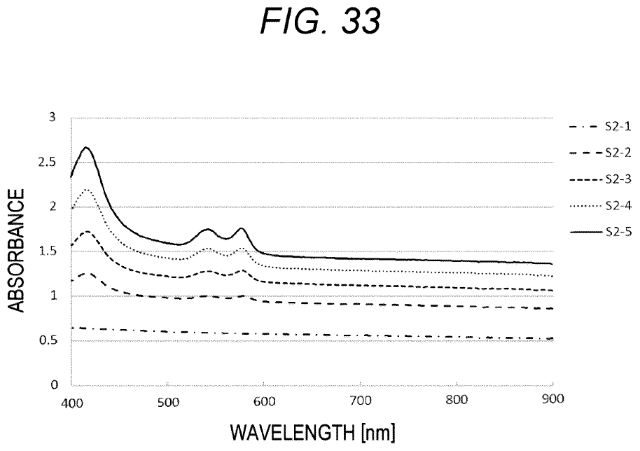

FIG. 32 illustrates an absorbance spectrum obtained by measuring each sample prepared for Verification experiment 1 with a double beam spectrophotometer, by using a cell having an optical path length of 2 mm. FIG. 33 illustrates an absorbance spectrum obtained by measuring each sample prepared for Verification experiment 2 with a double beam spectrophotometer, by using a cell having an optical path length of 2 mm.

### (3) Calculation Example

As one example, an example is shown in which the WBC concentration is calculated after correction on the RBC basis from a measurement result of Sample 5 in Verification experiment 1. From the measurement result of FIG. 32,

$$Absorbance at 420 [ nm ] : A 420 = 2.41$$

$$Absorbance at 700 [ nm ] : A 700 = 1.34$$

$$Absorbance difference = 2.41 - 1.34 = 1.07$$

Substituting this into Calibration curve (2) on the RBC basis expressed by the above Equation (13), the following is obtained:

$$\begin{matrix}R B C / W B C ratio = 4.51 \times absorbance {difference}^{0.669} \\ = 4.51 \times {1.07}^{0.669} \\ = 4.74\end{matrix}$$

Therefore, it was found that the RBC/WBC ratio of Sample 5 was 4.74. The RBC/WBC ratio at the time of creating Calibration curve (1) was 0.436, and how much the absorbance increased when the RBC/WBC ratio 0.436 becomes the RBC/WBC ratio 4.74 was calculated. The WBC concentration is different between the sample at the time of creating Calibration curve (1) and Sample 5. Therefore, in order to compare both with the same base, Calibration curve (3) on the RBC basis expressed by the above Equation (15) was used. Substituting each of the RBC/WBC at the time of creating Calibration curve (1) and the RBC/WBC ratio of Sample 5 into Calibration curve (3), the following is obtained:

$$Absorbance ( R B C / W B C ratio : 0.436 ) = ( - 1.93 \times {10}^{- 2} ) \times {0.436}^{2} + ( 3.53 \times {10}^{- 1} ) \times 0.436 + ( 2.74 \times {10}^{- 2} ) = 0.178$$

$$Absorbance ( R B C / W B C ratio : 4.74 ) = ( - 1.93 \times {10}^{- 2} ) \times {4.74}^{2} + ( 3.53 \times {10}^{- 1} ) \times 4.74 + ( 2.74 \times {10}^{- 2} ) = 1.27$$

The difference Ad is 1.09, and absorbance when the RBC/WBC ratio of Sample 5 is the same as that of Calibration curve (1) can be obtained by subtracting 1.09 from actually measured absorbance.

$$A 700 - Ad = 1.34 - 1.09 = 0.25$$

Therefore, by substituting this value into Calibration curve (1) as described below, the WBC concentration after correction could be calculated.

$$W B C concentration : C [ cells / mL ] = 4.64 \times {10}^{7} \times absorbance - 2.67 \times {10}^{6} = 4.64 \times {10}^{7} \times 0.25 - 2.67 \times {10}^{6} = 8.93 \times {10}^{6} [ cells / mL ]$$

(* The reason why the result of the calculation example slightly deviates from the value in Table 7 is that, in the calculation described above, calculation is performed with three significant digits in each stage, and a series of calculation in Table 7 is performed at once in Excel (registered trademark) which is software of Microsoft Corporation of the United States)

### (4) Measurement Results

The measurement results of Verification experiment 1 are shown in the following Table 7, and the measurement results of Verification experiment 2 are shown in the following Table 8. An error of the measurement results by the turbidity method was calculated assuming that an actual measurement value by the cell counter was regarded as positive.

TABLE 7

| Sample No. | | | | S2-1 | S2-2 | S2-3 | S2-4 | S2-5 |
|---|---|---|---|---|---|---|---|---|
| RBC/WBC ratio | | | | 0.56 | 1.77 | 2.34 | 3.82 | 4.40 |
| HGB/WBC [pg/cells] | | | | 26.9 | 60.5 | 87.4 | 123 | 149 |
| WBC | Actual measurement value | | | 1.79E+07 | 1.88E+07 | 1.75E+07 | 1.67E+07 | 1.68E+07 |
| concentration | [cells/mL] by cell counter | | | | | | | |
| | Measurement | Measured | No correction | 1.74E+07 | 3.41E+07 | 4.50E+07 | 5.43E+07 | 5.94E+07 |
| | by turbidity | value | Correction | 1.13E+07 | 1.35E+07 | 1.23E+07 | 9.63E+06 | 8.89E+06 |
| | method | [cells/ | on RBC basis | | | | | |
| | | mL] | Correction | 1.93E+07 | 2.14E+07 | 1.97E+07 | 1.65E+07 | 1.58E+07 |
| | | | on HGB basis | | | | | |
| | | Error | No correction | −2.67% | 81.5% | 157% | 225% | 254% |
| | | | Correction | −37.1% | −28.0% | −29.5% | −42.3% | −47.1% |
| | | | on RBC basis | | | | | |
| | | | Correction | 7.77% | 13.9% | 12.5% | −0.91% | −5.89% |
| | | | on HGB basis | | | | | |

TABLE 8

| Sample No. | | | | S2-1 | S2-2 | S2-3 | S2-4 | S2-5 |
|---|---|---|---|---|---|---|---|---|
| RBC/WBC ratio | | | | 0.58 | 1.66 | 2.34 | 3.51 | 4.68 |
| HGB/WBC [pg/cells] | | | | 26.0 | 56.6 | 85.7 | 125 | 162 |
| WBC | Actual measurement value | | | 2.03E+07 | 2.02E+07 | 1.96E+07 | 1.71E+07 | 1.66E+07 |
| concentration | [cells/mL] by cell counter | | | | | | | |
| | Measurement | Measured | No correction | 2.15E+07 | 3.77E+07 | 4.76E+07 | 5.53E+07 | 6.14E+07 |
| | by turbidity | value | Correction | 1.27E+07 | 1.17E+07 | 1.05E+07 | 8.62E+06 | 7.14E+06 |
| | method | [cells/ | on RBC basis | | | | | |
| | | mL] | Correction | 2.07E+07 | 1.93E+07 | 1.77E+07 | 1.55E+07 | 1.42E+07 |
| | | | on HGB basis | | | | | |
| | | Error | No correction | 5.71% | 86.9% | 143% | 224% | 270% |
| | | | Correction | −37.6% | −42.2% | −46.3% | −49.6% | −57.0% |
| | | | on RBC basis | | | | | |
| | | | Correction | 2.19% | −4.28% | −9.80% | −9.28% | −14.3% |
| | | | on HGB basis | | | | | |

In Sample S2-1 having the smallest RBC contamination rate, a reverse rotation phenomenon occurs. However, in both experiments, magnitude of the error was generally the largest in a case of “no correction”, the second largest in a case of “correction on RBC basis”, and the smallest in a case of “correction on HGB basis”.

Focusing on the presence of the RBC ghost in a sample derived from blood and considering the measurement principle of the hemacytometer, accuracy of correction can be improved by correcting absorbance on the basis of HGB instead of RBC. As can be seen from Calibration curve (2) and the graphs (FIG. 26 to 29) at the time of creating Calibration curve (3), a determination coefficient R2 of the approximate curve is larger in a case of the HGB basis than the RBC basis, and variations in data is further suppressed.

The present technology may also have the following configurations.

[1]

A concentration measurement device including:
an absorbance measurement unit configured to measure absorbance in a plurality of different wavelength regions for a mixed liquid in which different liquids are mixed; and
a control unit configured to correct a concentration of at least one liquid on the basis of absorbance in the plurality of different wavelength regions.
   [2]

The concentration measurement device according to [1], in which the absorbance measurement unit measures absorbance in two wavelength regions.

[3]

The concentration measurement device according to [1] or [2], in which the absorbance measurement unit measures absorbance in a spectrum.

[4]

The concentration measurement device according to any one of [1] to [3], in which particles contained in one of the different liquids are different from particles contained in another of the different liquids.

[5]

The concentration measurement device according to any one of [1] to [4], in which each of the different liquids is a suspension or a solution.

[6]

The concentration measurement device according to any one of [1] to [5], in which the mixed liquid includes a white blood cell suspension and a red blood cell suspension.

[7]

The concentration measurement device according to any one of [1] to [6], in which the control unit calculates a mixing ratio of a liquid mixed in the mixed liquid, from absorbance in the plurality of different wavelength regions.

[8]

The concentration measurement device according to any one of [1] to [7], in which the control unit corrects the absorbance on the basis of a mixing ratio of a liquid mixed in the mixed liquid.

[9]

The concentration measurement device according to any one of [1] to [8], further including: a calibration curve creation unit configured to create a calibration curve by using a calibration-curve-creation mixed liquid in which a component is same as a component of the mixed liquid and a concentration of each mixed liquid is known.

[10]

The concentration measurement device according to [9], in which the calibration curve creation unit creates a calibration curve for obtaining a concentration of at least one mixed liquid, on the basis of absorbance in a specific wavelength region by using the calibration-curve-creation mixed liquid in which a concentration of the at least one liquid is known.

[11]

The concentration measurement device according to [9] or [10], in which the calibration curve creation unit creates a calibration curve for obtaining a mixing ratio of a liquid mixed in the mixed liquid, on the basis of a difference in absorbance in the plurality of different wavelength regions.

[12]

The concentration measurement device according to any one of [9] to [11], in which the calibration curve creation unit creates a calibration curve for obtaining absorbance on the basis of a mixing ratio of a liquid mixed in the mixed liquid.

[13]

The concentration measurement device according to any one of [1] to [12], further including:
a light source configured to emit light;
a light-transmissive container inside of which a fluid containing the mixed liquid flows; and
a detection unit configured to detect light having passed through the container.
   [14]

The concentration measurement device according to [13], in which
the light source includes: a first light source configured to emit light having a first wavelength; and
a second light source configured to emit light having a second wavelength different from the first wavelength.
   [15]

A concentration measurement method including:
an absorbance measuring step of measuring absorbance in a plurality of different wavelength regions for a mixed liquid in which different liquids are mixed; and
a correction step of correcting a concentration of at least one liquid on the basis of absorbance in the plurality of different wavelength regions.
   [16]

The concentration measurement method according to [15], in which
the concentration measurement method further includes: a calibration curve creating step of creating a calibration curve by using a calibration-curve-creation mixed liquid in which a component is same as a component of the mixed liquid and a concentration of each mixed liquid is known.

### REFERENCE SIGNS LIST

1 Concentration adjustment device
2 Liquid container 2
3 Hollow fiber module
4 Absorbance measurement unit
5 Waste liquid container
6 Control unit
10 CONCENTRATION MEASUREMENT DEVICE
31 Hollow fiber membrane
32 Outer cylinder
41 First light source
42 First optical system
43 Tube holder
44 Third lens
45 Light shielding plate
46 Fourth lens
47 Fifth lens
48 Detection unit
49 Second light source
50 Sixth lens
51 First shutter
52 Second shutter 52
53 Dichroic mirror
71 to 75 Pipe
421 First lens
422 Second lens
431 Tube groove
432 Through hole
433, 434 Recess
451 Opening
1000 Computer
1050 Bus
1100 CPU
1200 RAM
1300 ROM
1400 HDD
1450 Program data
1500 Communication interface
1550 External network
1600 Input/output interface
1650 Input/output device
6100 Specimen analyzer
6101 Light irradiation unit
6102 Detection unit
6103 Information processing unit
6104 Sorting unit
C Flow channel
L1 Mixed liquid
L2 Waste liquid
P Particles
PO1 to PO3 Pump
S Specimen
TB Tube
V1 to V3 Valve

1. A concentration measurement device comprising:
an absorbance measurement unit configured to measure absorbance in a plurality of different wavelength regions for a mixed liquid in which different liquids are mixed; and
a control unit configured to correct a concentration of at least one liquid on a basis of absorbance in the plurality of different wavelength regions.

2. The concentration measurement device according to claim 1, wherein the absorbance measurement unit measures absorbance in two wavelength regions.

3. The concentration measurement device according to claim 1, wherein the absorbance measurement unit measures absorbance in a spectrum.

4. The concentration measurement device according to claim 1, wherein particles contained in one of the different liquids are different from particles contained in another of the different liquids.

5. The concentration measurement device according to claim 1, wherein each of the different liquids includes a suspension or a solution.

6. The concentration measurement device according to claim 1, wherein the mixed liquid includes a white blood cell suspension and a red blood cell suspension.

7. The concentration measurement device according to claim 1, wherein the control unit calculates a mixing ratio of a liquid mixed in the mixed liquid, from absorbance in the plurality of different wavelength regions.

8. The concentration measurement device according to claim 1, wherein the control unit corrects the absorbance on a basis of a mixing ratio of a liquid mixed in the mixed liquid.

9. The concentration measurement device according to claim 1, further comprising: a calibration curve creation unit configured to create a calibration curve by using a calibration-curve-creation mixed liquid in which a component is same as a component of the mixed liquid and a concentration of each mixed liquid is known.

10. The concentration measurement device according to claim 9, wherein the calibration curve creation unit creates a calibration curve for obtaining a concentration of at least one mixed liquid, on a basis of absorbance in a specific wavelength region by using the calibration-curve-creation mixed liquid in which a concentration of the at least one liquid is known.

11. The concentration measurement device according to claim 9, wherein the calibration curve creation unit creates a calibration curve for obtaining a mixing ratio of a liquid mixed in the mixed liquid, on a basis of a difference in absorbance in the plurality of different wavelength regions.

12. The concentration measurement device according to claim 9, wherein the calibration curve creation unit creates a calibration curve for obtaining absorbance on a basis of a mixing ratio of a liquid mixed in the mixed liquid.

13. The concentration measurement device according to claim 1, further comprising:
a light source configured to emit light;
a light-transmissive container inside of which a fluid containing the mixed liquid flows; and
a detection unit configured to detect light having passed through the container.

14. The concentration measurement device according to claim 13, wherein
the light source includes: a first light source configured to emit light having a first wavelength; and
a second light source configured to emit light having a second wavelength different from the first wavelength.

15. A concentration measurement method comprising:
an absorbance measuring step of measuring absorbance in a plurality of different wavelength regions for a mixed liquid in which different liquids are mixed; and
a correction step of correcting a concentration of at least one liquid on a basis of absorbance in the plurality of different wavelength regions.

16. The concentration measurement method according to claim 15, wherein the concentration measurement method further includes: a calibration curve creating step of creating a calibration curve by using a calibration-curve-creation mixed liquid in which a component is same as a component of the mixed liquid and a concentration of each mixed liquid is known.
